(19) United States
(12) Patent Application Publication
(10) Pub. No.: US 2018/0114594 A1
(43) Pub. Date: Apr. 26, 2018

(54) PATIENT-CENTRIC HEALTH RECORD SYSTEM AND RELATED METHODS

(71) Applicant: LUC BESSETTE, MONTREAL (CA)

(72) Inventors: LUC BESSETTE, MONTREAL (CA); YVES LE BORGNE, MONTREAL (CA)

(21) Appl. No.: 15/568,217

(22) PCT Filed: Apr. 13, 2016
(86) PCT No.: PCT/CA2016/050424

§ 371 Date: Oct. 20, 2017

Related U.S. Application Data
(60) Provisional application No. 62/150,013, filed on Apr. 20, 2015.

(51) Int. Cl. G16H 10/60; G16H 50/30; G06F 17/30

(52) U.S. Cl. CPC G16H 10/60; G06Q 10/1095; G06F 17/30864; G16H 50/30

(57) ABSTRACT

A patient-centric electronic health record (EHR) system is provided. The patient-centric EHR system allows for a patient to have centralized storage of his/his health records. The health records associated with the patient may be obtained from multiple sources in an EHR network, where the EHR network comprises multiple nodes storing health records associated with the patient. By obtaining the health records associated with the patient from multiple sources allows for a patient to have a centralized storage of his/her health records. Also, a centralized storage of health records associated with a patient also allows for the patient to have certain level of control over his/his health records, such as providing his/her health records to a third-party.

### FIELD OF THE INVENTION

The invention generally relates to electronic health record systems and in particular to patient-centric health record systems and related methods.

### BACKGROUND

Medicine has been for a long time a top down activity, where all activities are dictated by the capabilities and restrictions of care providers, and where the patient ends up being a passive voice in the decisions and chain of events. The doctor has the knowledge and the office, the patient has the waiting room. The patient's medical information is gathered and stored in the doctor's or hospital record. The patient must wait to get results and then wait again for a doctor that is part of his health management organization to be available. Pertinent medical data are the product of medical interventions and patient driven medical data is often not deemed contributive. Physicians are doing medical research—patients are studied.

The data and the power are currently in the hands of the few who are collecting the information. Cloaked with the screen of confidentiality, the information is not even shared nor aggregated in a cooperative way. For instance, health records associated with a patient may be stored at various locations, such as at an electronic health record (EHR) system associated with a physician, as part of a government regulated or state-owned health record system, pharmacies, health maintenance organizations (HMOs) and the like. A problem with such EHR systems is that a patient's health records are dispersed across multiple sources (e.g., multiple computer systems) and are not easily accessible if a patient wants a comprehensive record of his/her medical history.

As a patient's health records are located at multiple sources, this can further be a problem if a patient visits a new physician outside of the patient's regular physician's office and/or receives care outside of his normally accessible care delivery network, as the new physician does not have a complete understanding of the patient's medical history.

Moreover, any medical information regarding the new physician's assessments of the patient is not added to patient's file at the regular physician's office and/or the file that is part of the patient's normally accessible care delivery network.

Another problem with electronic patients' health records is that as these records may contain sensitive information, the communication of the health records should be done in way that confidentiality is maintained.

A further problem with patients' health records being located at multiple sources is that if third-parties (e.g., agents) would like access to patient information for various reasons they are required to obtain consent from the patient for each source and then access each of the various sources that store patients' health records separately to obtain a more complete medical history of the patients. Obtaining consent from patients from multiple sources and then accessing multiple sources can be tedious especially where patient information is desired for a large group of patients. Moreover, research institutes everywhere are using home-grown, institute specific tools to garner patient consent, and track both the patient and the patient's data as the patient moves from clinical procedure to clinical procedure.

In addition, when patients gets medical results done (e.g., tests, lab work, imaging, etc.) they are typically not informed if their medical results have been processed, completed or reviewed, unless their physician contacts them. This often leaves patients in a state of uncertainty and anxiety regarding their medical results and/or the completion status.

A patient may also make use of various portable and wearable devices, which measure various physical conditions. The data from these various devices are typically stored in a distributed computing environment (e.g., the “cloud”) which is separate from the other sources containing health records associated with the patient, which results in yet another source of health records associated with the patient.

In light of the above, there is a need in the industry for providing an improved EHR system and improved methods relating to EHR systems that alleviates, at least in part, the deficiencies with existing EHR systems.

### SUMMARY

In accordance with a first broad aspect, a patient-centric electronic health record (EHR) system is provided. The patient-centric EHR system may be able to obtain and/or receive various health records associated with one or more patients from one or more EHR networks that includes one or more nodes. The nodes in the EHR network may store health records associated with patients thereon. By accessing the EHR network, which may be done via a practitioner's EHR system, the health records associated with patients stored in the EHR network may be duplicated at least in part to the patient-centric EHR system.

In accordance with a specific and non-limiting example of implementation of the first broad aspect, a patient via a patient's computing entity may access the patient-centric EHR system to have access to his/her health record.

In accordance with a specific and non-limiting example of implementation of the first broad aspect, a health related determination mechanism is provided for allowing a patient's health record to be processed by the patient-centric EHR system to derive a health related determination.

In accordance with a specific and non-limiting example of implementation of the first broad aspect, a third-party access mechanism is provided for allowing a third-party to access and/or process a patient's health record.

In accordance with a specific and non-limiting example of implementation of the first broad aspect, an anonymized health record access mechanism is provided for providing anonymized health records to third-parties.

In accordance with a specific and non-limiting example of implementation of the first broad aspect, the patient-centric EHR system is provided for sharing a patient's health record among a care support group.

In accordance with a second broad aspect, a method for populating an electronic health record system with health records associated with respective patients is provided. The method includes: (a) querying a centralized health record network storing health records about multiple patients, the querying being performed in the context of a physician dispensing medical services to a first patient of the multiple patients, the querying including accessing the health record of the first patient; (b) copying some or all of the information in the health record of the first patient to the electronic health record system to create health record for the first patient in the electronic health record system; (c) repeating steps (a) and (b) for a second patient of the multiple patients; and (d) where the physician dispenses medical services to only a subset of patients of the multiple patients.

In accordance with a specific and non-limiting example of implementation of the second broad aspect, the method includes providing an account to the first patient such that the first patient can access the health record associated therewith on the electronic health record system.

In accordance with a third broad aspect, a method for providing health records associated with a patient to an electronic health record system is provided. The method includes: receiving an indication for a request to obtain the health records associated with the patient; requesting the health records associated with the patient from an electronic health record network comprising a plurality of nodes, the health records associated with the patient being distributed amongst the plurality of nodes of the health record network; obtaining the health records associated with the patient, in response to requesting the health records associated with the patient from the electronic health record network; and transmitting the health records associated with the patient to an electronic health record system for centralized storage of the health record.

In accordance with a fourth broad aspect, a method for providing at least one computing device associated with a patient a notice that medical results associated with the patient have been received at an office associated with a physician is provided. The method includes: receiving from an electronic record system associated with the physician an indication that medical results associated with the patient have been received at the office associated with the physician; transmitting to a computing entity associated with the patient an first notice that medical information is available; receiving a request for the medical information from the computing entity associated with the patient, the request including authentication information; and if the authentication information is associated with the patient, transmitting to the computing entity associated with the patient a second notice, the second notice indicating that medical results associated with the patient have been received at the office associated with the physician.

In accordance with a fifth broad aspect, a method for providing medical results associated with a patient to a computing device associated with the patient is provided. The method includes: receiving medical results associated with the patient from an electronic record system associated with a physician; transmitting to a computing entity associated with the patient an first notice that medical information is available; receiving a request for the medical information from the computing entity associated with the patient, the request including authentication information; and if the authentication information is associated with the patient, transmitting to the computing entity associated with the patient a second notice, the second notice comprising the medical results associated with the patient.

In accordance with a sixth broad aspect, a method for making a health related determination is provided. The method includes: monitoring data obtained from one or more sensors measuring a physical condition of a patient, the monitoring of the data from the one or more sensors being done over a plurality of time points; storing the data from the one or more sensors in association with medical records associated with the patient; and processing the data from the one or more sensors against the medical records associated with the patient to make a health related determination, the medical records being obtained from a plurality of electronic health record systems.

In accordance with a seventh broad aspect, a method for providing a third-party with health records associated with a patient is provided. The method includes: receiving a request to provide the third-party with the health records associated with the patient; authenticating the request with a computing device associated with the patient; and providing a third-party computing device with access to the health records associated with the patient.

In accordance with a eighth broad aspect, a method of providing health records to an agent is provided. The method includes: receiving a request for a plurality of health records meeting a specific criteria from a third-party computing entity associated with the agent; obtaining a plurality of health records meeting the specific criteria; and providing to the third-party computing entity the plurality of health records.

These and other aspects of the invention will now become apparent to those of ordinary skill in the art upon review of the following description of embodiments of the invention in conjunction with the accompanying drawings.

### BRIEF DESCRIPTION OF THE DRAWINGS

A detailed description of embodiments of the invention is provided below, by way of example only, with reference to the accompanying drawings, in which:

FIG. 1A illustrates a block diagram of an example patient-centric electronic health record (EHR) system connected to various other EHR systems and to a computing entity associated with a patient in accordance with an embodiment of the invention.

FIG. 1B illustrates a block diagram of an example EHR network comprising a central EHR system in accordance with an embodiment of the invention.

FIG. 1C illustrates a block diagram of an example EHR network comprising various EHR systems and a computing entity associated with a patient connected to the EHR network in accordance with an embodiment of the invention.

FIG. 2A illustrates a block diagram of a patient-centric EHR system in accordance with a specific example of implementation.

FIG. 2B illustrates a block diagram of a physician's EHR system in accordance with a specific example of implementation.

FIGS. 3A to 3C illustrates examples of data records in accordance with embodiments of the inventions.

FIG. 4A illustrates a flowchart of a process, which may be implemented by the physician's EHR system of FIGS. 1A-1C in accordance with a specific example of implementation.

FIG. 4B illustrates a flowchart of a process, which may be implemented by the patient-centric EHR system in accordance with a specific non-limiting example of implementation.

FIG. 4C illustrates a flowchart of a process, which may be implemented by central EHR system in accordance with a specific non-limiting example of implementation.

FIG. 5A illustrates a block diagram of an EHR network comprising a single EHR system in accordance with a specific non-limiting example of implementation.

FIG. 5B illustrates a block diagram of an EHR network comprising a plurality of EHR systems accessible by a server in accordance with a specific non-limiting example of implementation.

FIG. 5C illustrates a block diagram of an EHR network comprising a plurality of EHR systems in accordance with a specific non-limiting example of implementation.

FIG. 6 illustrates a block diagram of an example of a patient-centric EHR system that is able to provide patients with medical results upon authorization in accordance with an embodiment of the invention.

FIG. 7A illustrates a flowchart of a process for determining if the patient-centric EHR system should be notified of receipt of medical results at a physician's office in accordance with a specific non-limiting example of implementation.

FIG. 7B illustrates a flowchart of a process for transmitting medical results to the patient-centric EHR system in accordance with a specific non-limiting example of implementation.

FIG. 8A illustrates a flowchart of a process for sending a notice to the patient's computing entity in accordance with a specific non-limiting example of implementation.

FIG. 8B illustrates a flowchart of a process for sending medical results associated with the patient to the patient's computing entity in accordance with a specific non-limiting example of implementation.

FIG. 9 illustrates a block diagram of an example of a patient-centric EHR system that is able to receive and obtain auxiliary medical related information in accordance with an embodiment of the invention.

FIG. 10 illustrates a flowchart of a process for making a health related determination in accordance with a specific non-limiting example of implementation.

FIG. 11 illustrates a block diagram of an example of a patient-centric EHR system that provides health records associated with a patient to a third-party EHR system in accordance with an embodiment of the invention.

FIG. 12A illustrates a flowchart of a first process for providing one or more health records associated with a patient to a third-party in accordance with a specific non-limiting example of implementation.

FIG. 12B illustrates a flowchart of a second process for providing one or more health records associated with a patient to a third-party in accordance with a specific non-limiting example of implementation.

FIG. 13A illustrates a block diagram of an example of a patient-centric EHR system that provides health records associated with a patient to a third-party EHR system in accordance with another embodiment of the invention.

FIG. 13B illustrates a flowchart of a third process for providing one or more health records associated with a patient to a third-party in accordance with a specific non-limiting example of implementation.

FIG. 14 illustrates a block diagram of an example of a patient-centric EHR system for providing patient anonymized health records to a third-party computing entity in accordance with an embodiment of the invention.

FIG. 15 illustrates a flowchart of a process for providing patient anonymized health records to a third-party in accordance with a specific non-limiting example of implementation.

FIGS. 16A, 16B, 16C, 16D, 16E and 16F illustrate specific and non-limiting examples of information that may be displayed on a graphical user interface (GUI) of a patient's computing entity in accordance with specific examples of implementation.

FIGS. 17A, 17B and 17C illustrate specific and non-limiting examples of notices in accordance with specific examples of implementation.

FIG. 18 illustrates a block diagram of an example patient-centric EHR system connected to various other EHR systems and to a computing entity associated with a patient in accordance with an embodiment of the invention.

FIGS. 19A, 19B, 19C, 19D and 19E illustrate specific and non-limiting examples of information that may be displayed on a graphical user interface (GUI) of a physician's computing entity in accordance with specific examples of implementation.

FIGS. 20A and 20B illustrate flowcharts of processes for creating a care support group in accordance with a specific non-limiting example of implementation.

FIG. 21 illustrates an example of a data structure for the care support group in accordance with a specific non-limiting example of implementation.

It is to be expressly understood that the description and drawings are only for the purpose of illustrating certain embodiments of the invention and are an aid for understanding. They are not intended to be a definition of the limits of the invention.

### DETAILED DESCRIPTION

Current medical systems are neither patient-centric, nor patient-driven, nor patient-aware. The scope of this disclosure is to reverse the data ownership paradigms, empowering the patient himself in key data collection and access decisions, and set the ground for a transformation by which the medical system is patient-centric, patient-driven and patient aware.

For example, whenever new data is stored in an electronic health record or a health information system, a proxy could alert the key holder. Informed that his laboratory result may be commented by his practitioner before a set time, he may have the possibility to pursue it after that delay or, better, read the comment and initiate a course of action accordingly. If his doctor is not available, he could consult a third party doctor and give him for a period of time access to his medical information through his private key. Accessing this information, the third party doctor may carry an on-site or telemedicine consultation with the patient.

A patient-centric health record infrastructure may allow for the process of separating the nominative (name, address, phone number or any identifying tag) from the content-rich non-nominative information (rich medical data). A patient-centric health record infrastructure may also allow the ability to ask individually for permission to use non-nominative data for research, such as allowing for the ability to identify query-driven cohort of patients. Such cohorts could be established from patient records by allowing an agent to find all patients matching certain criteria, and eventually request from each patient permission to access the health records required to conduct further specific health analyses establishing in this way patient membership in a specific research topic. The patient may have the ability to permit/deny his discoverability for cohorts, and also assuming he has authorized discoverability, permit/deny use of his health record in the fulfillment of the intent of the cohort. By definition, a cohort may be non-nominative to protect the privacy of the patient.

Data derived from cohorts of patients is very seldom paid back by users, those being epidemiologists, pharmaceutical companies, biomedical companies, etc. Some users may pay a third party for databanks derived of patient data but patients themselves are for the most part ignored completely when one has to pay for collected data. With the embodiment of the invention, patients may repossess their data and may get some advantage of having their data used either by having a small share of profits derived by the use of their data or some other financial compensation.

These and other aspects should likely become more readily apparent to the person skilled in the art throughout this document.

### Patient-Centric EHR System

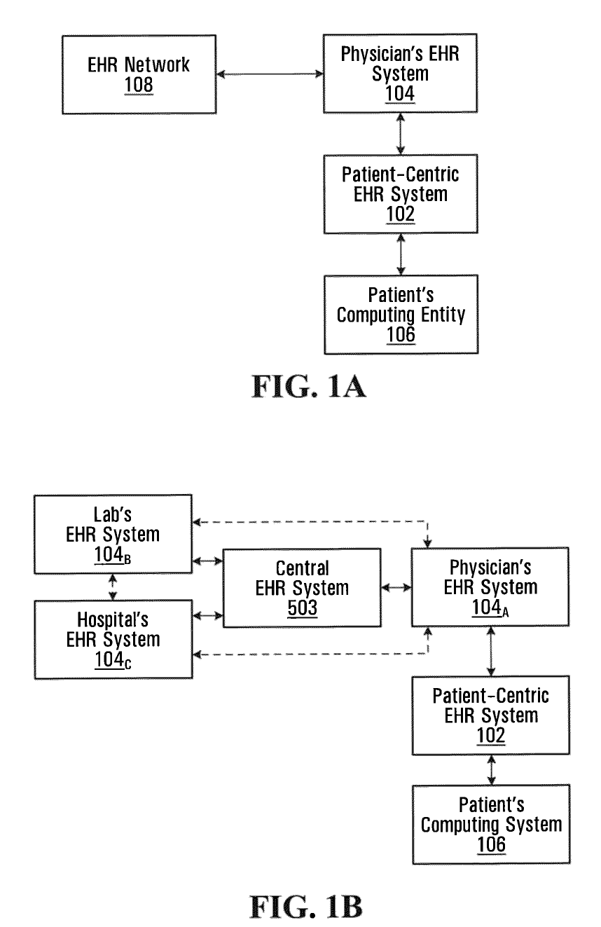

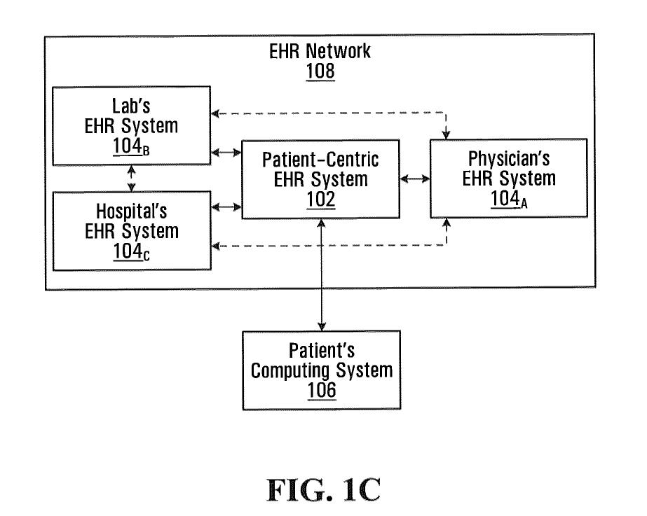

FIG. 1A illustrates a block diagram of an example patient-centric electronic health record (EHR) system 102 connected to various other EHR systems and to a computing entity associated with a patient in accordance with an embodiment of the invention. As illustrated the patient-centric EHR system 102 is connected to the patient's computing entity 106 (e.g., a computing entity associated with a patient) and to a physician's EHR system 104 (e.g., an EHR system associated with a physician) which is connected to an EHR network 108. Although in FIG. 1 only a single patient-centric EHR system 102, a single physician's EHR systems 104, a single EHR network 108 and a single patient's computing entity 106 are shown, this is for illustration purposes only, as one or more patient-centric EHR systems, one or more physician's EHR systems, one or more EHR networks and one or more patient's computing entities may be used in implementing the various embodiments described herein.

The various connections between the patient-centric EHR system 102, the patient's computing entity 106, the physician's EHR system 104 and/or the EHR network 108 may include one or more connections over one or more data networks (e.g., the Internet, local area network (LAN), or a wide area network (WAN), cellular networks, other wired or wireless networks and/or any other suitable data network). In other words, the patient-centric EHR system 102, the patient's computing entity 106, the physician's EHR systems 104 and/or the EHR network 108 may be nodes in one or more data networks linked by communication paths. The various connections between the patient-centric EHR system 102, the patient's computing entity 106, physician's EHR systems 104 and/or the EHR network 108 may allow for the communication (e.g., transmission and/or reception) of various information and/or data between the various systems and/or devices. The various information and/or data exchanged may be notices, data records, health records, medical records and/or medical related information. The term “health record” may be used throughout this document to refer to a single data record, health record, medical record and/or other medical or health related information and/or data and the term “health records” may be used throughout this document to refer to a plurality of data records, health records, medical records and/or other medical or health related information and/or data. Reference to a single “health record” in this document may include reference to a plurality of “health records”.

In general terms, the patient centric EHR system 102 allows for a patient to have centralized storage of his/her health records by obtaining the health records associated with the patient from various sources and also allows for the patient to have some control over his/her health records. In other words, “patient centric” refers to the patient centric EHR system 102 possibly offering a patient with fine-grained control over visibility of health records associated with the patient. The control of the health records associated with the patient may include who can view the health records and/or what health records can be viewed. In particular, some health records may have a ‘for your eyes only’ nature, and be annotated as such. In that case, the patient centric EHR system 102 may only transmit the information when the patient has identified himself strongly as being the recipient. Furthermore, the ‘for your eyes only’ annotation may cause the health record being becoming ‘invisible’ after a set delay after the patient has seen the content. For instance, the health record may become ‘invisible’ by revoking any and all access rights that could exist for the record, for other users other than the patient himself/herself. Also, the record may become destroyed from volatile memory and then may only be viewed again if the patient himself/herself requests the records after a two-factor authentication at the device (e.g., finger print and user identifier). These and other aspects of the patient centric EHR system 102 should likely become more readily apparent to the person skilled in the art throughout this document.

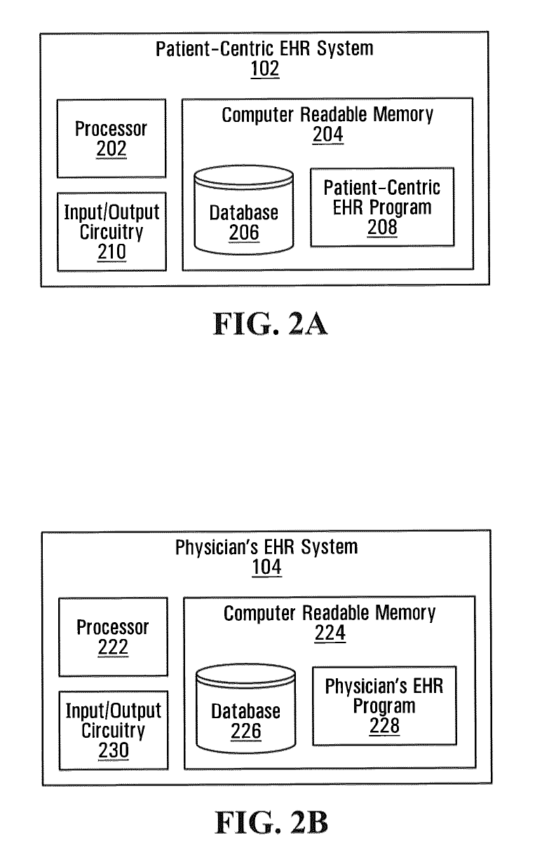

FIG. 2A illustrates a block diagram of the patient-centric EHR system 102 in accordance with a specific example of implementation. In general terms, the patient-centric EHR system 102 may be a single computing entity or a distributed computing environment (e.g., server arrangements) that stores health records associated with patients. As illustrated, the patient-centric EHR system 102 comprises at least one processor 202, input/output circuity 210 and at least one computer readable memory 204 comprising at least one database 206 and a patient-centric EHR program 208 (e.g., program code, application software, etc). The at least one processor 202, input/output circuity 210 and at least one computer readable memory 204 may be connected by various data buses and in the case of a distributed computing environment may be interconnected by one or more data networks. The at least one database 206 stores one or more health records associated with a patient and stores a plurality of health records associated with a plurality of patients. The patient-centric EHR program 208 comprises program code which when executed by the at least one processor 202 causes the patient-centric EHR system 102 to perform various operations. The input/output circuity 210 may be used to connect the patient-centric EHR system 102 to one or more data networks and to other peripheral devices (e.g., keyboard, mouse, monitor/display, printer and/or any other suitable device).

FIG. 2B illustrates a block diagram of the physician's EHR system 104 (e.g., an EHR system associated with a physician and/or any other health related practitioner) in accordance with a specific example of implementation. In general terms, the physician's EHR system 104 may be a single computing entity or a distributed computing environment (e.g., server arrangements) that stores health records associated with patients. For instance, the physician's EHR system 104 may be of the type that is typically found in physician's office for maintaining health records of patients but with additional functionality as should likely become more readily apparent to the person skilled in the art throughout this document. As illustrated, the physician's EHR system 104 comprises at least one processor 222, input/output circuity 230 and at least one computer readable memory 224 comprising at least one database 226 and at least one physician's EHR program 228. The at least one processor 222, input/output circuity 230 and at least one computer readable memory 224 may be connected by various data buses and in the case of a distributed computing environment may be interconnected by one or more data networks. The at least one database 226 stores one or more health records associated with a patient and stores a plurality of health records associated with a plurality of patients. The physician's at least one EHR program 228 comprises program code which when executed by the processor 222 causes the physician's EHR system 104 to perform various operations. The input/output circuity 230 may be used to connect the physician's EHR system 104 to one or more data networks and to other peripheral devices (e.g., keyboard, mouse, monitor/display, printer and/or any other suitable device).

The patient's computing entity 106 (e.g., a computing entity associated with a patient) may be any portable or non-portable computing device, such as a cell-phone, a tablet, a smart watch, a laptop/notebook computer, a computer or any other suitable computing device. The patient's computing entity 106 may comprise program code that when executed cause the patient's computing entity 106 to perform various operations, such as application software. In some cases, the patient's computing entity 106 runs application software that is associated with the patient-centric EHR system 102. The application software may include client-side program code used to interact with the patient-centric EHR system 102 having server-side program code. For example, client-side program code may include JavaScript that invokes AJAX quires to the server-side program code of the patient-centric EHR system 102. The patient's computing entity 106 may include a display/screen or is connect to a display/screen in which a graphical user interface (GUI) may be provided. The GUI may be conditioned based on the program code (e.g., application software) and/or various data received from the patient-centric EHR system 102.

The EHR network 108 may comprises one or more EHR systems. In general terms, an EHR system may be a single computing entity or a distributed computing environment (e.g., server arrangements) that stores health records associated with patients and the EHR system typically includes at least one processor, input/output circuity and computer readable memory including a database and program code. For instance, the one or more EHR systems of the EHR network 108 may be similar to the physician's EHR system 104 and/or the patient-centric EHR system 102 described elsewhere in this document. The one or more EHR systems of the EHR network 108 may include one or more systems such as: health maintenance organization's (HMO's) EHR system; other physicians' EHR systems; EHR systems associated with pharmacies; EHR systems associated with dentists; EHR systems associated with optometric; EHR systems associated with other medical facilities (e.g., laboratories such as testing and imagining labs); government regulated or state-owned health record system; and/or any other suitable system. By way of example, in Quebec, the government regulated health record system is the DSQ (Dossier de sante du QuÉbec) and is some embodiments, the EHR network 108 comprises the DSQ. As such, the EHR network 108 may include a central EHR system 503, as shown in FIG. 1B, implemented as a single computing entity or a distributed computing environment (e.g., server arrangements) that typically includes at least one processor, input/output circuitry and computer readable memory including a database and program code. It is appreciated that the central EHR system 503 may be a government regulated or state-owned EHR system or HMO's EHR system and/or any other suitable EHR system.

In some cases the EHR network 108 may comprise a medical information exchange (MIE) or a summary medical record system. MIEs and summary medical record systems are known in the art, such as the type disclosed in Canadian Patent No. 2,329,598 or PCT International Publication No. WO 2015/031980, both of which are incorporated herein by reference. In other words, the EHR network 108 may be implemented in a distributed nature in a data network including multiple nodes linked by communication paths. That is, the EHR network 108 may be implemented by one or more nodes in a data network linked by communication paths. As various implementation of the EHR network 108 are known in the art, the EHR network 108 does not need to be described in detail because such systems are well within the reach of a person skilled in the art.

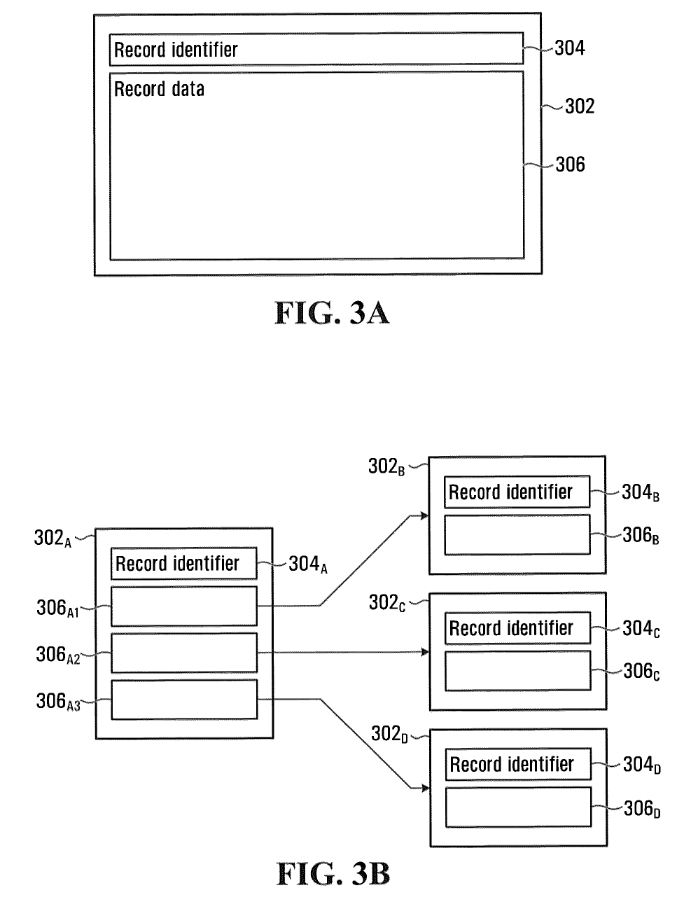

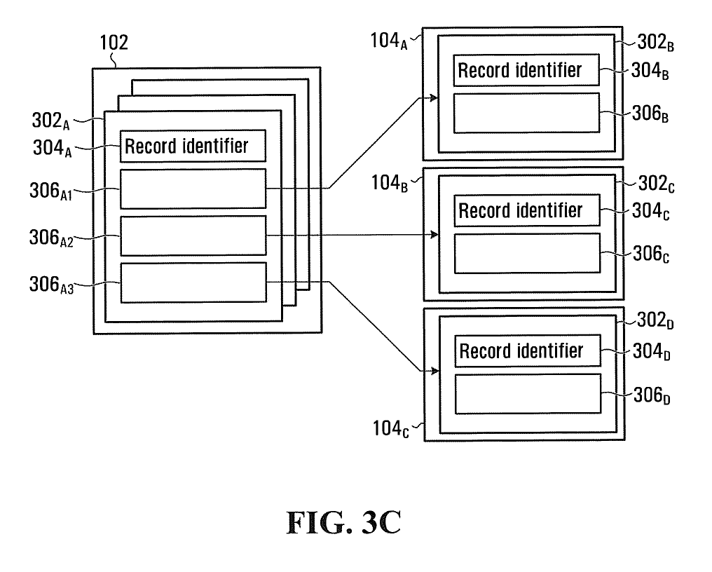

FIGS. 3A to 3C illustrate examples of data records 302 in accordance with embodiments of the invention. It will be understood that the representation is conceptual to facilitate the understanding of the way the information is organized and it is not intended to be limiting in terms of how the data conveying that information is being structured or how the information is shown on a display device.

The data record 302 includes a record identifier 304 and record data 306. The record identifier 304 is used to identify the specific data record 302 and to distinguish it from the data records of other patients. The record data 306 is associated with the record identifier 304 such that information relevant to a specific patient identified by the record identifier 304 is stored in the record data 306 of the data record 302. The record data 306 may be physically stored in one centralized location or may be stored in a distributed fashion with a mechanism to link the various data components together so all the useful information can be retrieved when required. As such, the record data 306 may include data/information and/or may point to locations where data/information may be stored.

By way of example, FIG. 3B illustrates a data record 302_{A} having a record identifier 304_{A} and a plurality of records 306_{A} 306_{B} 306_{C} where each of the records 306_{A} 306_{B} 306_{C} includes a respective pointer pointing to respective ones of the data records 302_{B} 302_{C} 302_{D}. Each of the data records 302_{B} 302_{C} 302_{D} has a respective identifier 304_{B} 304_{C} 304_{D} and respective data record 306_{B} 306_{C} 306_{D}. It is appreciated that in these examples that the record data 306 may be a combination of data that is stored in one location and also includes links and/or pointers to data stored in a remote location, which can be accessed when required by downloading the data from the remote location pointed to by the one or more links. Although in FIG. 3A that the data record 302 is shown to only include a single record data 306, it is appreciated that the data record 302 may include a plurality of records of the type of the data record 306, such as shown in FIG. 3B.

By way of another example, FIG. 3C illustrates an example that is similar to that of FIG. 3B, as data record 302_{A} has a record identifier 304_{A} and a plurality of records 306_{A} 306_{B} 306_{C} where each of the records 306_{A} 306_{B} 306_{C} includes a respective pointer pointing to respective ones of the data records 302_{B} 302_{C} 302_{D}. However, as shown in FIG. 3C, the data records 302_{B} 302_{C} 302_{D} are respectively stored on separate EHR systems, namely, a physician's EHR system 104_{A}, a laboratory EHR system 104_{B} and a hospital EHR system 104_{C} (e.g., such as shown in FIG. 1C).

The data record 302 may be a health record of the type used (e.g., accessed, stored, obtained, communicated, transmitted and/or received) by the various systems and devices discussed in this document. As such, “health record” may be used to refer to a data record of the type of the data record 302 and “health records” may be used to refer to a plurality of data records of the type of data record 302. The record data 306 of the data record 302 may include links and/or pointers to other data records of the type of the data record 302 by pointing to these other data records' identifiers and where these other data records' record data includes the additional information associated with the data record 302. Reference to a single “data record” in this document may include reference to a plurality of “data records”. It is appreciated that the health records may be located at various nodes (e.g., various EHR systems and/or other suitable computer based system) in the EHR network 108.

In the embodiments where the data record 302 is a health record, the data record 302 may include information such as: prescribed medication, delivered medication, laboratory results, pathology reports, consultation reports, imaging reports and images themselves, ECG (electrocardiogram) reports or the images themselves, surgical or procedure reports with or without images, allergies or medication intolerances, hospitalization summaries, physician summaries and/or any other suitable information. The data record 302 may also include patient identification information such as the patient's name, address, date of birth, health card number, primary physician, the issuing physician that prescribed the medication or medial test, and/or any other suitable information. The information stored in the data record 302 is not limited to the non-exhaustive list given above, a person skilled in the art would understand that other types of patient health and/or medical information may also be stored in the data record 302 in the various embodiments disclosed in this document.

In some embodiments, where the data record 302 is a health record stored on the EHR network 108, the data record 302 may include a summary component that includes information such as summaries of: Administrative Data, Permanent Biological Data, Significant Antecedents, Current Medical Conditions, Biological Data, Prescribed and/or Delivered Medications, Laboratory Results, Pathology Reports, Consultation Reports, Imaging Reports and Images, ECG reports and/or ECG Images, Surgical or Procedure Reports, Allergies and/or Medication Intolerances, Hospitalization Summaries or Physician Summaries. Furthermore, each summary may be associated with a pointer, which points to more complete information provided by the summary. By way of a specific and non-limiting example, the ECG reports summary may list pointers to where the ECG images are actually stored. Similarly, different laboratory reports, images, prescribed prescriptions, and so forth, may be at different nodes of the data network and the summary records contains links that point to the different nodes in the network that store the complete information. As such, the person skilled in the art would clearly understand that any number of combinations of different types of data records where some data records are stored on various nodes (e.g., various EHR systems) of the EHR network 108 could exist. As various implementations of the data records are known in the art, data records do not need to be described in detail because they are well within the reach of a person skilled in the art.

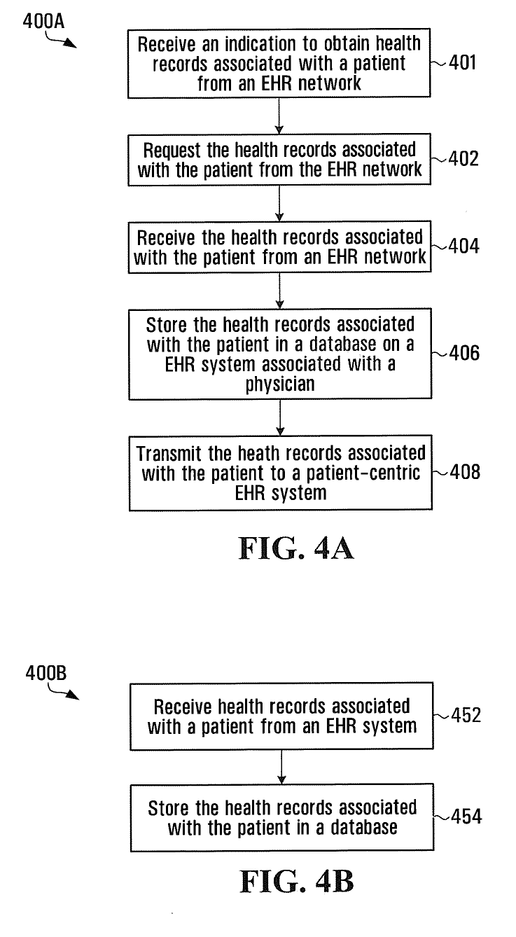

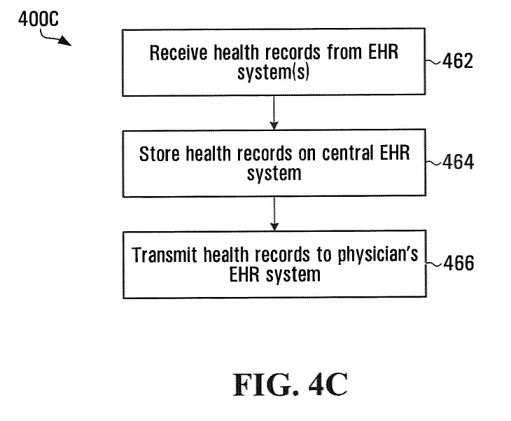

FIG. 4A illustrates a flowchart of a process 400A, which may be implemented by the physician's EHR system 104 in accordance with a specific example of implementation. At step 401 the physician's EHR system 104 receives an indication (e.g., a request) to obtain health records associated with a patient from the EHR network 108. In some cases, at step 401, the patient-centric EHR system 102 initiates the process to obtain health records of the patient from the EHR network 108 by transmitting a request to the physician's EHR system 104. In these cases, the patient provides authorization to the patient-centric EHR system 102 to retrieve the patient's health records from various sources. This may include the patient creating an account with the patient-centric EHR system 102, which may be done via the patient's computing entity 106. In some cases, the patient uses a web browser running on the patient's computing entity 106 to connect to a website associated with the patient-centric EHR system 102. In other cases the patient may download an application that runs on the patient's computing entity 106 in order to connect to the patient-centric EHR system 102 to create an account. In the account creation process, the patient may have to provide information about himself/herself, such information may include: name, address, date of birth, place of birth, health card number, social insurance number, email, user name, password, biometric identifier and/or any other suitable information. The patient-centric EHR system 102 may then use the information received from the patient to authenticate that the person making the account is in fact that person. Regardless of the means that the patient creates an account with the patient-centric EHR system 102, upon creation of an account and authorization from the patient, the patient-centric EHR system 102 may initiate the process of obtaining the health records associated with the patient. Once the account is created, the patient-centric EHR system 102 may maintain a record associated with the patient, where the record includes an account identifier (e.g., an account name/number, health card number, email and/or any suitable identifier) and a credential which may be used to authenticate the patient (e.g., a password, biometric information and/or any other suitable credential). It is appreciated that each account may have more than one account identifier and/or more than one credential.

In other cases, at step 401, the indication to obtain health records associated with a patient from an EHR network 108 may be initiated by the physician, such as when a patient is at the physician's office and requests for his/her health records to be added to the patient-centric EHR system 102. Similar to the case above, an account may be created at the patient-centric EHR system 102. For example, the physician may create an account with the patient-centric EHR system 102 upon verbal confirmation from the patient. In some cases, the patient may already have an account created with the patient-centric EHR system 102 and visits the physician so that the physician via the physician's EHR system 104 may request the health records associated with the patient from the EHR network 108. Similar to how the patient has an account with the patient-centric EHR system 102, physicians may also have accounts with the patient-centric EHR system 102. For example, a physician may have an account with the patient-centric EHR system 102 so that the physician may communicate via the physician's EHR system 104 with the patient-centric EHR system 102. In such case, the patient-centric EHR system 102 would have accounts associated with various physicians, where each account includes at least one identifier and at least one credential, such that a physician via the physician's EHR system 104 may then use the identifier and/or the credential in connecting to the patient-centric EHR system 102.

In some instances, the health records of the patient available in the EHR network 108, such as a HMO and/or government maintained and ran EHR network and/or system, may be extracted when the physician consults the health records of the patent by logging into the EHR network. When the physician accesses the health records, the data in the records, which is owned by the patient, can be legitimately stored into the patient-centric EHR system 102. In this fashion, the patient-centric EHR system 102 is populated with medical data every time the HMO and/or government managed EHR system is accessed by a physician. Over time, the HMO and/or government managed EHR system may be completely replicated into the patient-centric EHR system 102. In other cases, the patient-centric EHR system 102 may replicate the index (e.g., a list of pointers that point to specific records in the nodes of the EHR network 108) of the EHR network 108. In other words, meta-information may be replicated without replicating the data itself. The EHR network 108 is not limited to HMO and/or government maintained and ran EHR networks and/or systems, but may be any suitable EHR network(s) and/or system(s) managed by one or more suitable organizations.

The record replication mechanism, which is performed every time the physician accesses the government and/or HMO managed EHR system 108, ensures that the information available in the patient-centric EHR system 102 is maintained up to date. If changes have been made to the EHR record in the HMO and/or government-managed system, those changes are automatically carried over into the patient-centric EHR system 102.

At step 402, the physician's EHR system 104 requests the health records associated with the patient from the EHR network 108. This may include the physician's EHR system 104 connecting to the EHR network 108, in which the physician has an account with. For example, the physician may provide his/her username and password and/or hardware token such that the physician's EHR system 104 is able to connect to the EHR network 108. The process of EHR systems connecting to the EHR network 108 (e.g., MIE) is known in art, for example as is disclosed in PCT International Publication No. WO 2015/031980, the contents of which are incorporated herein by reference. As various means for connecting to the EHR network 108 are known in the art, the means for connecting by the physician's EHR system 104 to the EHR network 108 does not need to be described in detail because such means are well within the reach of a person skilled in the art. The requests for the health records associated with the patient from the EHR network 108 may include an identifier associated with the patient, which the EHR network 108 may use to obtain the health records associated with the patient. The requests for the health records associated with the patient from the EHR network 108 may also include an identifier of the physician's EHR system 104 making the request.

Even more specifically, the physician accesses the government-managed or HMO-managed EHR system during a consultation, which access includes downloading the medical data to the physician's computer to display it on the physician's display. The software that manages the replication of the medical data makes a copy of the information sent from the EHR government-managed or HMO-managed system to the patient-centric EHR system. The software can also be designed to parse the information made available when the file access was requested to determine if additional data needs to be obtained such as to obtain a complete copy of the patient record. For example, if the medical data stored in the government-managed EHR system includes a summary portion that uses links allowing access to additional medical data, the software is configured to parse the file and recognize the links and invoke them such that the additional medical information is also sent by the HMO and/or government-managed EHR system to the physician. That additional medical information is then also copied and integrated into the patient-centric EHR system 102.

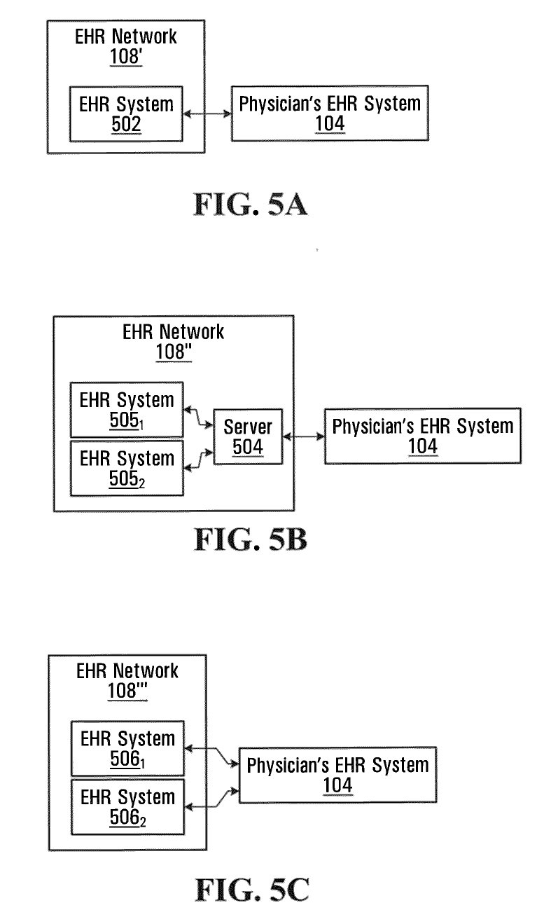

FIG. 5A illustrates a block diagram of an EHR network 108′ comprising a single EHR system 502 in accordance with a specific non-limiting example of implementation. The EHR network 108′ is a specific non-limiting example of implementation of the EHR network 108 (discussed elsewhere in this document). In this example, the single EHR system 502 stores the health records associated with the patient. In the context of this example, at step 402, the physician's EHR system 104 requests the health records associated with the patient from the single EHR system 502. In turn, the single EHR system 502 transmits the health records associated with the patient to the physician's EHR system 104.

FIG. 5B illustrates a block diagram of an EHR network 108″ comprising a plurality of EHR systems 505₁ 505₂ accessible by a server 504 in accordance with a specific non-limiting example of implementation. The server 504 refers to one or more computing entities (e.g., machines) on which a server program runs that waits for requests from other computing entities (e.g., physician's EHR system 104) and responds to them. The server 504 may be a record aggregation system. The EHR network 108″ is a specific non-limiting example of implementation of the EHR network 108 (discussed elsewhere in this document). In this example, the EHR network 108″ comprises a plurality of EHR systems 505₁ 505₂, such that the health records associated with the patient is distributed amongst the plurality of EHR systems 505₁ 505₂. In other words, the health records associated with the patient are distributed amongst the EHR network 108″ such that at least part of the health records associated with the patient are stored on each of the EHR systems 505₁ 505₂. In the context of this example, at step 402, the physician's EHR system 104 requests the health records associated with the patient from the centralized server 504 associated with the EHR network 108″. In turn, the server 504 communicates with the plurality of EHR systems 505₁ 505₂ to obtain the requested health records associated with the patient, which may include the server 504 querying the plurality of EHR systems 505₁ 505₂. Then the server 504 transmits the health records associated with the patient to the physician's EHR system 104. The server 504 in some cases may be an EHR system which stores at least part of the health records associated with the patient. More specifically, the server 504 may be the central EHR system 503.

FIG. 5C illustrates a block diagram of the EHR network 108′″ comprising a plurality of EHR systems 506₁ 506₂ in accordance with a specific non-limiting example of implementation. The EHR network 108′″ is a specific non-limiting example of implementation of the EHR network 108 (discussed elsewhere in this document). In this example, the EHR network 108′″ comprises a plurality of EHR systems 506₁ 506₂, such that the health records associated with the patient is distributed amongst the plurality of EHR systems 506₁ 506₂. In other words, the health records associated with the patient is distributed amongst the EHR network 108′″ such that at least part of the health records associated with the patient is stored on each of the EHR systems 506₁ 506₂. In the context of this example, at step 402, the physician's EHR system 104 requests health records associated with the patient from each of the plurality of EHR systems 506₁ 506₂. In turn, each of the plurality of EHR systems 506₁ 506₂ transmits health records associated with the patient to the physician's EHR system 104.

The various EHR systems 502, 505₁ 505₂ 506₁ and 506₂ may be of the type of EHR system that is discussed elsewhere in this document. Although in both FIG. 5B and FIG. 5C two EHR systems 505₁ and 505₂ or 506₁ and 506₂ are shown, the EHR network 108 may comprise more than two EHR systems. The configuration of the EHR network 108 illustrated in FIGS. 5A, 5B and 5C is intended to not be limiting and various other configurations of the EHR network 108 are possible in other embodiments. Although in FIGS. 5A, 5B and 5C the physician's EHR system 104 is shown connecting directly to the EHR network 108, in other examples the patient-centric EHR system 102 may connect directly to the EHR network 108. Any communication with the EHR network 108 from other systems and/or devices as discussed in this document may be communication with any of the systems and/or devices that are included in the EHR network 108, for example, such as those shown in FIGS. 5A-5C.

Turning back to FIG. 4A, at step 404, the physician's EHR system 104 receives the health records associated with the patient from the EHR network 108. In other words, at step 404, the physician's EHR system 104 obtains the health records associated with the patient, in response to requesting the health records associated with the patient from the EHR network 108.

At step 406, the health records associated with the patient may then be stored in the physician's EHR system 104. For example, the obtained health records associated with the patient may be stored in the database 226 of the physician's EHR system 104 in association with the patient. In some cases, the database 226 of the physician's EHR system 104 may have additional medical information relating to the patient. In other words, at least part of the health records associated with the patient may be stored in the database 226 of the physician's EHR system 104 prior to the physician's EHR system 104 making the request to the EHR network 108 to obtain the remaining parts of the health records associated with the patient. In other cases, the physician's EHR system 104 and the EHR network 108 may have at least in part duplicate information relating to the health records associated with the patient.

Then at step 408, the health records associated with the patient is transmitted to the patient-centric EHR system 102. The patient-centric EHR system 102 receives the health records associated with the patient and stores these health records in one or more records in the database 206 in association with the patient. In other words, the health records associated with the patient may be stored in a manner that is associated with the patient's account. Such a configuration, may allow for the patient to access his/her health records by connecting to the patient-centric EHR system 102 (e.g., via a web browser or an application running on the patient's computing entity 106) and log into the patient's account. By allowing the patient to access his/her health records on the patient-centric EHR system 102 the patient may be able to control various aspects of his/her health records. For instance, the patient's account may have a profile associated with it, where the profile has various settings, parameters and/or options that the patient can configure. Moreover, where the health records associated with the patient are obtained from multiple sources (e.g., multiple nodes/systems of the EHR network 108) and then stored in the patient-centric EHR system 102, this may allow for a centralized storage of the health records associated with the patient.

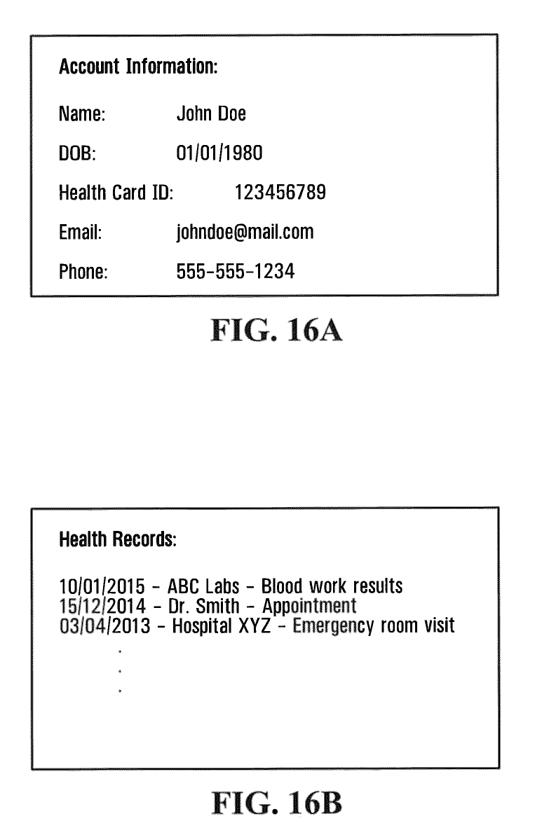

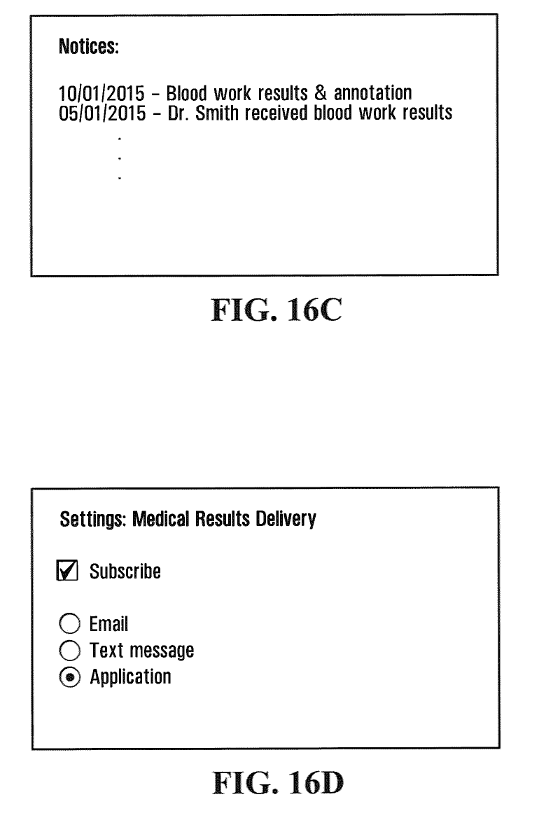

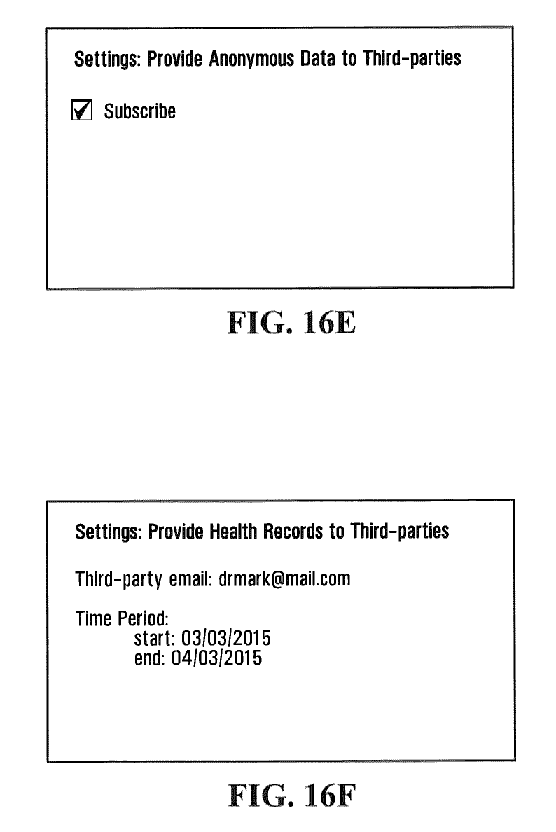

FIGS. 16A-16F illustrate specific and non-limiting examples of information that may be displayed on the GUI of the patient's computing entity 106 in accordance with specific and non-limiting examples of implementation. FIG. 16A illustrates an example of account information associated with the patient. FIG. 16B illustrates a plurality of medical records obtained from multiple sources. As shown in this example, the plurality of medical records includes laboratory results from ABC Labs, annotations from an appointment with Dr. Smith and annotations from a hospital visit to Hospital XYZ. The patient via the patient's computing entity 106 may be able to connect to the patient-centric EHR system 102 after step 408 of process 400A and view the plurality of medical records associated with the patient (e.g., as shown in FIG. 16B). The patient may also be able receive various notices from the patient-centric EHR system 102, as shown in FIG. 16C and discussed elsewhere in this document. The patient may also be able to control various aspects of the plurality of medical records stored on the patient-centric EHR system 102 and associated with the patient for example as is shown in FIGS. 16D, 16E and 16F.

FIG. 4B illustrates a flowchart of a process 400B which may be implemented by the physician's EHR system 104 in accordance with a specific example of implementation. In this example, the physician's EHR system 104 registers with the EHR network 108 this may include registering with the central EHR system 503 (or the server 504, as shown in FIG. 5B) or one or more EHR systems (e.g., such as those shown in FIGS. 5A and 5C). The registration process may include the physician's EHR system 104 providing to the EHR network 108, central EHR system 503 and/or various EHR systems a list of patients, where the list of patients include patients that the physician provides medical services to. The list of patients may include for each patient on the list the patient's name, date of birth, health identifier number and/or any other suitable information. Once registered, the physician's EHR system 104 is subscribed to receive records associated with the list of provided patients. That is, when the EHR network 108, central EHR system 503 and/or various EHR systems updates a health record associated with a patient, the updated record and/or information associated with the updated record is transmitted to the physician's EHR system 104. At step 452, the physician's EHR system 104 receives the health record and then, at step 454, stores the health record in association with the patient's record on the physician's EHR system 104. In other words, the physician's EHR system 104 automatically receives the health records for all of its patients, regardless of where a specific patient is registered with the patient-centric EHR system 102. The physician's EHR system 104 may then transmit the records of the patients that are registered with the patient-centric EHR system 102 to the patient-centric EHR system 102, in a similar manner as discussed elsewhere in this document.

FIG. 4C illustrates a flowchart of a process 400C which may be implemented by the central EHR system 503 in accordance with a specific example of implementation. The process 400C is now discussed with reference to FIG. 1B. At step 462, the central EHR system 503 receives from one or more EHR systems (e.g., the physician's EHR system 104_{A}, the laboratory EHR system 104_{B} and the hospital EHR system 104, etc.) one or more health records. The received health records may be transmitted to the central EHR system 503 upon request from the central EHR system 503 to the one or more EHR systems or may automatically be transmitted to the central EHR system 503 from the one or more EHR systems, for example, when a record is updated or based on a period of time (e.g., on a daily basis). The central EHR system 503 may process the received records and, at step 464, store the records in association with the corresponding patients that the records correspond thereto in a database of the central EHR system 503. At step 466, the central EHR system 503 may then transmit health records on the central EHR system 503 to the physician's EHR system 104. For instance, the central EHR system 503 may have a list of registered EHR systems that are to receive patient records. The central EHR system 503 may then process in response to receipt of the records from step 462 and if any of those records are associated with a patient that is associated with the subscribing physician's EHR system 104, then the central EHR system 503 transmits those records to the subscribing physician's EHR system 104.

Although in the embodiments illustrated above, the patient-centric EHR system 102 receives the health records associated with the patient via the physician's EHR system 104, in other cases the patient-centric EHR system 102 may be connected directly to the EHR network 108 to request and obtain the health records associated with the patient. For instance, the patient-centric EHR system 102 may request the health records associated with the patient directly from the EHR network 108 in a similar fashion to that discussed in regard to step 402; the patient-centric EHR system 102 may receive the health records associated with the patient directly from the EHR network 108 in a similar fashion to that discussed in regard to step 404; and the patient-centric EHR system 102 may store the health records associated with the patient in a similar fashion to that discussed in regard to step 406.

For example, as shown in FIG. 1C, the patient-centric EHR system 102 may be part of the EHR network 108 such that it is connected to various EHR systems (e.g., a physician's EHR system 104_{A}, a laboratory EHR system 104_{B} and a hospital EHR system 104_{c}) of the EHR network 108. In other cases, the patient-centric EHR system 102 may be connected to and communicates with the central EHR system 503, where the central EHR system 503 is connected to and communicates with various EHR systems (e.g., the physician's EHR system 104_{A}, the laboratory EHR system 104_{E} and the hospital EHR system 104_{c}).

It is appreciated that FIGS. 1A to 1C are examples of possible configurations and interconnections of the patient-centric EHR system 102 and the EHR network 108 and that other configuration are possible in other embodiments.

The patient-centric EHR system 102 may also maintain a record of the various physicians' EHR systems that the patient has medical records associated with.

The technique for populating an EHR system such as the physician's EHR system 104 and/or the patient-centric EHR system 102 may be implemented in various ways. One technique for populating an EHR system with health records associated with respective patients includes querying the centralized EHR network 108 storing health records about multiple patients. The querying may be performed in the context of a physician dispensing medical services to a first patient of the multiple patients. For example, this may take place when the first patient is at the physician's office seeking a medical consult (e.g., an appointment with the physician). This medical consult may be to seek medical advice/services and/or may be for the purpose of populating the EHR system with health records associated with the first patient. The querying in this example includes accessing the health record of the first patient from the centralized EHR network 108. It may also include accessing the health record of the first patient from physician's EHR system 104, for example if the physician's EHR system 104 has additional health records about the first patient that are not on the centralized EHR network 108. This technique could then include copying some or all of the information in the health record of the first patient on the centralized EHR network 108 to the EHR system to create a health record for the first patient in the electronic health record system. This copying may include copying some or all of the information in the health record of the first patient on the centralized EHR network 108 to the physician's EHR system 104 and/or the patient-centric EHR system 102. The physician's EHR system 104 may transmit some or all of the information in the health record of the first patient on the centralized EHR network 108 to the patient-centric EHR system 102 for storage.

The querying and copying step may then be repeated for a second patient of the multiple patients to access and copy the health record of the second patient from the centralized EHR network 108. The querying and copying step may then be repeated numerous times such as for a third patient, a fourth patient and so on, of the multiple patients. For example, this may be done each time that the physician is dispensing medical services to a patient of the multiple patients.

It is appreciated that the physician may dispense medical services to only a subset of patients of the multiple patients. In other words, the centralized EHR network 108 stores health records about multiple patients and the physician may only access and copy the health records of the patients that the physician is dispensing medical services thereto. This is the case because the centralized EHR network 108 would typically also store health records associated with patients that are not patients of the physician but of other physicians.

By way of example, each time the physician dispenses medical services to the first patient, the querying of the centralized EHR network 108 may be repeated to access and copy any new health records associated with first patient to the EHR system. This may be consider as an update of the first patient's record on the EHR system based on any new records of the first patient on the centralized EHR network 108. In other cases, the querying of the centralized EHR network 108 may be done to access and update the first patient's health records on the EHR system upon request from the physician and/or the physician's EHR system 104. For example, when the physician desires to obtain the new health records associated with the first patient from the centralized EHR network 108 and/or when physician's EHR system 104 has been programmed to access the centralized EHR network 108 such as on a regular interval (e.g., daily, weekly, month basis and/or any other suitable timeframe). In even further cases, the centralized EHR network 108 may be programmed to push the new health records of the first patient to the electronic health record system such as on a regular interval (e.g., daily, weekly, month basis and/or any other suitable timeframe).

It is appreciated that the aforementioned electronic health record system that was populated with health records associated with respective patients could provide an account to the first patient such that the first patient can access the health record associated therewith on the electronic health record system. An account may also be provided to the second patient, and so forth. The account provided may be as discussed elsewhere in this document. This account may be an account to the physician's EHR system 104 and/or the patient-centric EHR system 102.

In some embodiments, the EHR network 108 may be implemented to include the use of block-chains. A block-chain is a distributed non-centralized database that maintains a continuously-growing list of data records that each refers to previous items on the list, which may be referred to as a ledger. More specifically, the block-chain includes blocks that hold timestamped data records. Each block also includes a hash of the prior block, linking the blocks together. The linked blocks form a chain, each additional block reinforcing those before it, thus giving the database type its name. As such, in this embodiment, instead of the EHR network 108 having a database management system a block-chain backed ledger system may be used. In this embodiment, various nodes would include at least a partial copy of the block chain.

It should be appreciated that the EHR network 108 may be a centralized or non-centralized network depending on the implementation of the EHR network 108.

Medical Results Delivery Mechanism

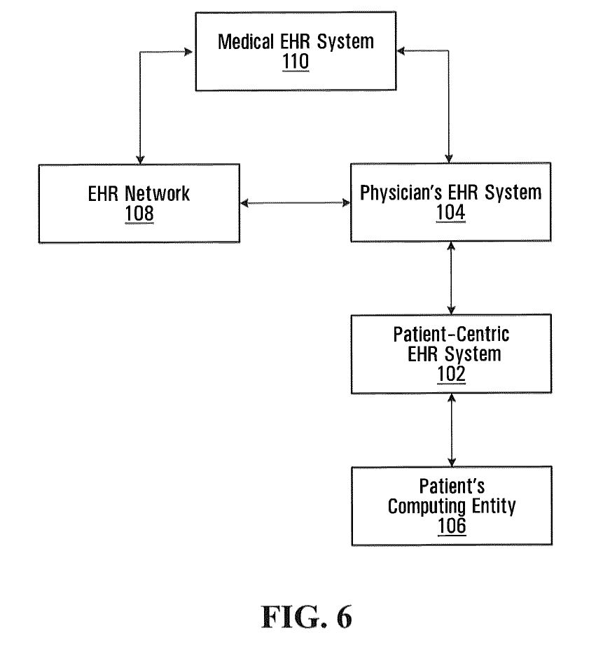

FIG. 6 illustrates a block diagram of an example of the patient-centric EHR system 102 that is able to provide patients with medical results upon authorization in accordance with an embodiment of the invention. As illustrated, the patient-centric EHR system 102 is connected to the patient computing entity 106 and to the physician's EHR systems 104 which is connected to EHR network 108. Also, a medical EHR system 110 is illustrated as being connected to both the EHR network 108 and to the physician's EHR systems 104. Although in the embodiment shown the medical EHR system is connected to both the EHR network 108 and the physician's EHR system 104, in other embodiments the medical EHR system 110 may only be connected to one of the EHR network 108 and physician's EHR system 104. It is appreciated that other connections are possible in other embodiments.

The various connections between the patient-centric EHR system 102, the patient's computing entity 106, the physician's EHR system 104, the medical EHR system 110 and/or the EHR network 108 may be similar to the various connections between systems and devices, as discussed elsewhere in this document. The various connections between the patient-centric EHR system 102, the patient's computing entity 106, the physician's EHR system 104, the medical EHR system 110 and/or the EHR network 108 may include one or more connections over one or more data networks. The various connections between the patient-centric EHR system 102, the patient's computing entity 106, the physician's EHR system 104, the medical EHR system 110 and/or the EHR network 108 may allow for the communication (e.g., transmission and/or reception) of various information and/or data between the various systems and/or devices. The various information and/or data exchanged may be a “notice”, “notices”, a “health record” and/or “health records” of the type discussed elsewhere in this document. In other words, the term “medical results” may refer to a notice, notices, a health record and/or health records.

The medical EHR system 110 may be one or more computer systems associated with a laboratory, testing facility, imaging facility or any other suitable facility that provides medical results associated with a test or image of a patient (e.g., an EHR system associated with a laboratory, testing facility and/or imaging facility). The medical result may include test results, such as blood test results, urine test results; imaging results, such as an X-ray image results or CAT (computerized axial tomography) scan results; CT (computerized tomography) scan; ECG images and/or reports; or any other suitable laboratory, imagining or test result and/or reports. The medical results may be provided in a health record associated with the patient.

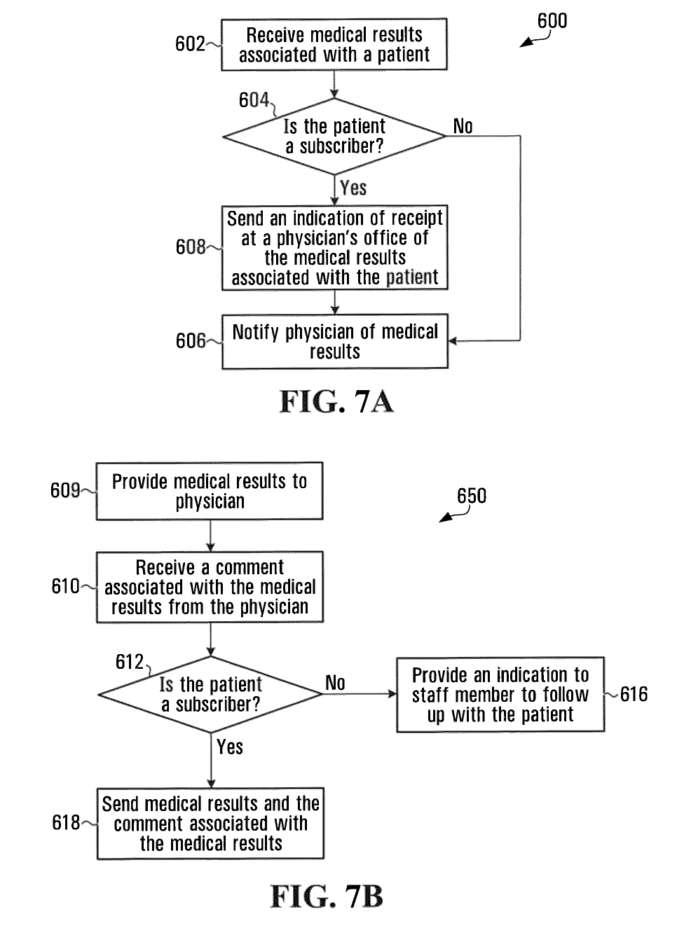

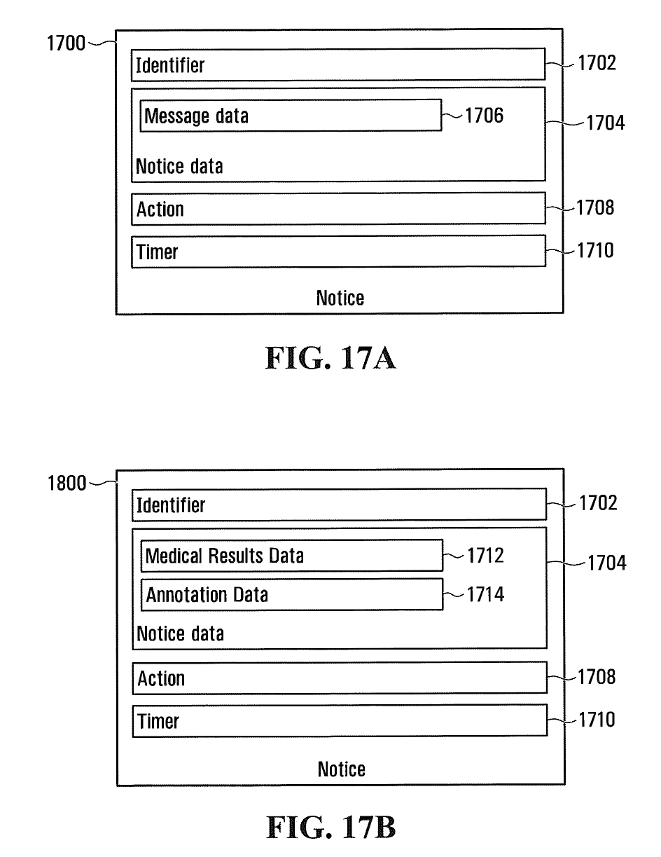

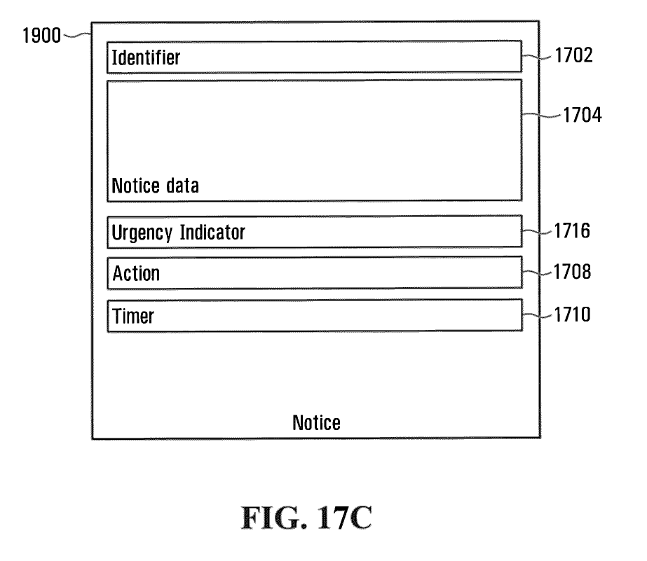

FIG. 7A illustrates a flowchart of a process 600, which may be implemented by the physician's EHR system 104 for determining if the patient-centric EHR system 102 should be notified of receipt of medical results at a physician's office in accordance with a specific non-limiting example of implementation. Prior to process 600, it is appreciated that a patient may have visited the physician and the physician requested that the patient get medical results done (e.g., testing or lab-work done). After the patient visits the facility to get the medical testing or lab-work done, the results are entered into a medical EHR system 110. The patient-centric EHR system 104 may receive an indication from the physician's EHR system 104 that medical testing or lab-work has been prescribed and the patient-centric EHR system 104 may send a notice to the patient's computing entity 106 to get medical results done. The patient-centric EHR system 104 may use this indication to setup a reminder notice if the patient does not get the medical testing or lab-work done within a certain timeframe. The notices are discussed elsewhere in this document, such as the discussion in association with FIGS. 17A and 17B.

At step 602, medical results associated with a patient are received at the physician's EHR system 104. At this step, the medical results may be received from the medical EHR system 110 or may be received from the EHR network 108 (e.g., the central EHR system 503 and/or any other EHR system). For instance, in some cases, the medical EHR system 110 may send the medical results to the EHR network 108 and the physician's EHR system 104 queries the EHR network 108 to obtain the medical results or the EHR network 108 transmits the medical results to the physician's EHR system 104 upon receipt from the medical EHR system 110. In other cases, the medical results may be provided in hardcopy and a user enters and/or scans the medical results into the EHR network 108 and/or physician's EHR system 104. Regardless of how the medical results are received, at this step the medical results may be stored in a health record associated with the patient.

At step 604, the patient information associated with the medical results is processed to see if the patient is a subscriber to the patient-centric EHR system 104 and/or a subscriber to a medical results delivery service. For example, prior to the physician's EHR system 104 receiving the medical results associated with the patient, the patient may have logged into his/her account and subscribed to the medical results delivery service. As such, the patient-centric EHR system 104 may manage a subscribing list of the patients that subscribe to the medical results delivery service. FIG. 16D illustrates a specific and non-limiting example of a profile page of the patient's account, which provides the options to subscribe to the medical results delivery service. As illustrated, the patient-centric EHR system 104 may also allow for the patient to select the means for receiving notices (e.g., email, text messages and application). In other cases, the patient may not have the option to select the mode for receiving notices; for example, in the case where the patient's computing entity 106 is running application software associated with the patient-centric EHR system 104, the notices may be received via the application software. Turning back to FIG. 7A, at step 604, the physician's EHR system 104 may query the patient-centric EHR system 104 to determine if the patient associated with the medical results is on the subscriber list or not. In this case, the patient-centric EHR system 102 maintains a subscriber list which lists the subscribers (e.g., various patients) and the physician's EHR system 104 may provide the patient-centric EHR system 104 with an identifier associated with the patient (e.g., a medical card number, an account identifier or any other suitable identifier) that can then be used by the patient-centric EHR system 104 to make a determination of whether the patient associated with the identifier is a subscriber or not. In other cases, the physician's EHR system 104 may query the patient-centric EHR system 102 for a subscriber list comprising identifiers associated with patients that are a part of the service and then can determine by processing the subscriber list against an identifier associated with the patient. In this case, if a match is found then the patient associated with the medical results is a subscriber and if a match is not found then the patient associated with the medical results is not a subscriber. Yet, in other cases, the physician's EHR system 104 may include in the medical records associated with the patient that the patient is a subscriber to the medical results delivery service. Regardless of how it is determined that the patient is a subscriber, at this step a determination is made (e.g., at the physician's EHR system 104 and/or the patient-centric EHR system 102) to determine whether a specific patient is a subscriber or not.

By way of example, the interaction between the physician's EHR system 104 and the patient-centric EHR system 102 to determine if a specific patient is a subscriber may be implemented as follows. The physician's EHR system 104 queries the patient-centric EHR system 102 by sending a request to see if the patient-centric EHR system 102 already has health records associated with a specific patient. The request may include providing a patient's name, gender, date of birth, date of death, social insurance number, a medical card identifier and/or any other suitable identifier. The patient-centric EHR system processes the request and in the case that a match is found (i.e., the patient-centric EHR system has health records associated with the requested patient), the patient-centric EHR system 102 may then communicate to the physician's EHR system 104 that the patient-centric EHR system 102 has health records associated with the requested patient and that this specific patient may be identified by a specific identifier (e.g., a proxy identifier). This specific identifier may be specific to the requesting physician's EHR system 104. In other words, each physician's EHR system in a plurality of physician's EHR system that may communicate with the patient-centric EHR system 102 would have a specific identifier for a specific patient, where the specific identifier is different for each of the physician's EHR systems. In the case that a match is not found, the patient-centric EHR system 102 may still provide the specific identifier for the requested patient and an indication that the patient-centric EHR system 102 currently does not have any health records associated with the requested patient. It is appreciated that by providing the specific identifier even though the patient-centric EHR system 102 does not currently have any health records associated with the requested patient that the patient-centric EHR system 102 may be later able to identify the patient when other physician's EHR systems try to request health records associated with the patient. Such a configuration between the patient-centric EHR system 102 and physician's EHR system 104 may allow for when the physician's EHR system 104 provides the patient-centric EHR system 102 with health records associated with the patient, that the physician's EHR system 104 provides the specific identifier of the patient along with an unique identifier associated with the physician's EHR system 104 (e.g., a doubleton).

If the patient is a subscriber to the service, then at step 608 an indication that the medical results have been received at the physician's office is transmitted from the physician's EHR system 104 to the patient-centric EHR system 102. At step 606, the physician's EHR system 104 may then notify the physician of the received medial results. If the patient is not a subscriber, then the physician's EHR system 104 may then notify the physician of the received medial results (step 606). In other cases, an optional note may be made in a record associated with the patient that an indication was not sent to the patient-centric EHR system 102. In other words, a note that the patient was not informed of receipt of the medical results at the physician's office. In other cases, when the patient is a subscriber, an optional note may be made that patient was informed that the medical results were received at the physician's office.

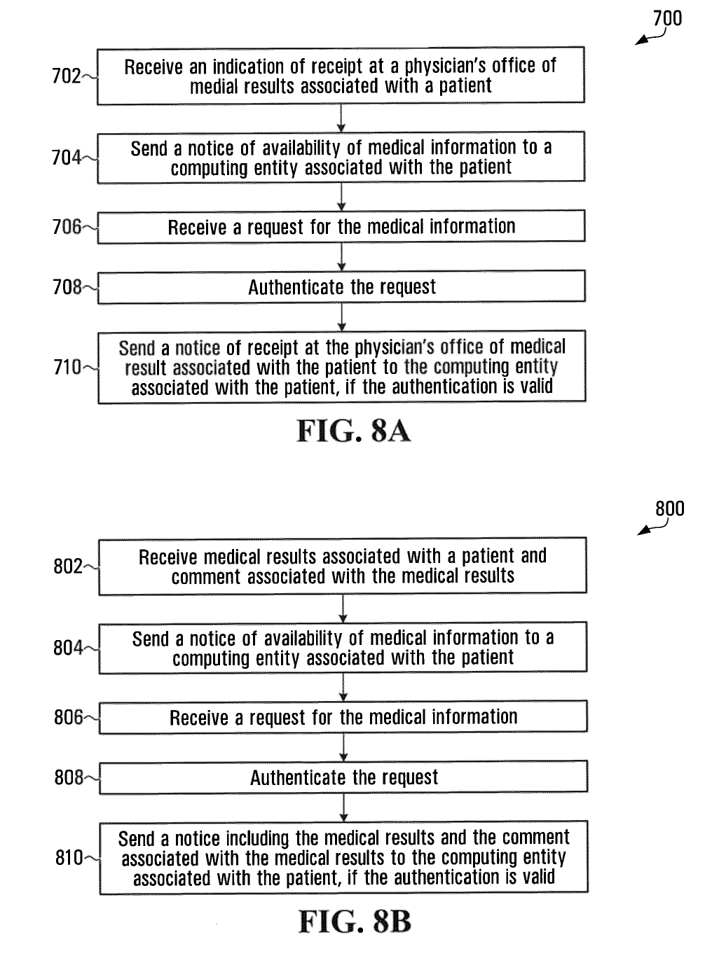

FIG. 8A illustrates a flowchart of a process 700 which may be implemented by the patient-centric EHR system 102 for sending a notice to the patient's computing entity 106 in accordance with a specific non-limiting example of implementation. At step 702, the patient-centric EHR system 102 receives from the physician's EHR system 104 an indication that that medical results associated with a patient that subscribes to the notification system of patient-centric EHR system 102 (i.e., a subscriber) have been received at the physician's office. Step 702 may take places after step 608 of process 600 and in general terms is an indication that medical results are available in the physician's EHR system 104 for review by the physician. At this step the patient-centric EHR system 102 may store the indication that that medical results associated with the patient have been received at the physician's office in a record associated with the subscribing patient. It is appreciated that there may be a distinction between a patient and a subscriber (or a subscribing patient), as the physician's EHR system 104 would typically have electronic health records associated with different patients, and the physician's EHR system 104 may provide the electronic health records to the patient-centric EHR system 102 regardless of whether the patient subscribes to the notifications of the patient-centric EHR system 102.

At step 704, the patient-centric EHR system 102 then sends a notice to the subscribing patient's computing entity 106 associated with the subscribing patient to notify the subscribing patient that medical information is available. The notice that medical information is available may depend on the type of delivery method that the subscribing patient desires to obtain notices by. For example, the subscribing patient may provide an email address to receive the notice in the form of an email; a cell-phone number to receive the notice as a text message. In other cases, where the subscribing patient's computing entity 106 runs an application associated with the patient-centric EHR system 102 and the application may provide the notice in the form of a pop-up box or window type notice or a red circle in a top corner of an icon associated with the application running on the subscribing patient's computing entity 106. Regardless of the means for providing the notice, the notice may only note that medical information is available for viewing at the patient-centric EHR system 102 and not provide any specific details regarding the medical information. In other words, patient intervention would typically be required to make the actual medial information visible to the user of the subscribing patient's computing entity 106, as discussed in the steps below.

At step 706 a request for the medical information from the subscribing patient's computing entity 106 is received and the request may include authentication information. In the cases where the notice is provided via email or text-message, the subscribing patient via the subscribing patient's computing entity 106 may login to the patient-centric EHR system 102 (e.g., through the use of a web browser) to obtain the medical information. For instance, the subscribing patient may provide a user account identifier and a password. In the cases where the notice is provided in the form of a pop-up box or window type notice in the application running on the subscribing patient's computing entity 106, the subscribing patient may provide a user account identifier, a password and/or biometric authentication information (e.g., a finger print via a finger print reader on the patient's computing entity 106) via the application. In other words, at this step the subscribing patient's computing entity 106 requests from the patient-centric EHR system 102 access to medical information by providing authentication information.

At step 708, the authentication information included in the request for the medical information is authenticated (e.g., by comparing the authentication information against a record associated with the patient that stores the credential information). Then at step 710 if the authentication information is associated with the subscribing patient, the patient-centric EHR system 102 then transmits a notice providing the medical information to the subscribing patient's computing entity 106. In this case, the medical information is a notice that medical results have been received at his/her physician's office but has not yet been reviewed.

In some cases, step 704 may be omitted from the process 700. In these cases, a notice of the availability of medical information is not transmitted to the subscribing patient's computing entity 106 and the patient-centric EHR system 102 waits for a request from the subscribing patient's computing entity 106.

In other cases, step 708 may be omitted from the process 700 and prior to step 704 the subscribing patient's computing entity 106 provides authentication information and if the authentication information is associated with the subscribing patient, then a notice may be sent at step 704. In other words, notices are not sent unless the subscribing patient has authenticated his/her patient's computing entity 106 to the patient-centric EHR system 102 in a manner that indicates he/she is currently using the device and would like notices to be received.

FIG. 7B illustrates a flowchart of a process 650 which may be implemented by the physician's EHR system 104 for transmitting medical results to the patient-centric EHR system 102 in accordance with a specific non-limiting example of implementation. After step 606 of the process 600, the medical results may be provided to a physician at step 609 of process 650. In other words, after the physician's EHR system receives the medical results associated with the patient, the physician is able to view the medical results and, at step 609, the medical results associated with the patient are provided to the physician. For example, the display/screen of the physician's EHR system 104 may provide the physician with the results and the physician may provide a comment (e.g., an annotation) via an input device such as a keyboard, mouse and/or touch screen. It is appreciated that the terms “comment” and “annotation” may be used interchangeably in this document, where appropriate, to refer to any annotation, comment, observation and/or explanation associated with a medical result. At step 610, the physician provides a comment regarding the medical results and the physician's EHR system 104 receives the comment and stores the comment in association with the medical results in a health record associated with the patient. At step 612, the physician's EHR system 104 then checks to see if the patient is a subscriber (step 612 of process 650 is similar to step 604 of process 600). If the patient is a subscriber, then at step 618 the medical results associated with the patient and the comment associated with the medical results for the patient are sent to the patient-centric EHR system 102 from the physician's EHR system 104 (step 610 of process 650 is similar to step 608 of process 600; however, instead of providing an indication of the medical results, the medical results themselves are transmitted). It is appreciated that transmission of the medical results may include the actual medical results and/or an explanation of the medical result, which may be transmitted in the form of an electronic health record. It is further appreciated that instead of transmission of the medical results that an explanation of the medical results may be transmitted instead. If the patient is not a subscriber, then at step 616, an indication would typically be provided to a staff member (e.g., nurse, receptionist, etc.) to follow-up with the patient (e.g., give them a call or send them an email) to book an appointment to come in and see his/her medical results, if appropriate in the circumstances. Optionally, a note may be made in the health record associated with the patient that the medical results was not sent to the patient-centric EHR system 102 to indicate that the patient was not notified of the medical results. In other cases, an optional note may be made in the health record associated with the patient that the patient has been informed of the medical results.

FIG. 8B illustrates a flowchart of a process 800 which may be implemented by the patient-centric EHR system 102 for sending medical results associated with the subscribing patient to the subscribing patient's computing entity 106 in accordance with a specific non-limiting example of implementation. At step 802 the patient-centric EHR system 102 receives from the physician's EHR system 104 medical results associated with the subscribing patient. Step 802 may take places after step 618 of process 650 and in general terms the medical results associated with the subscribing patient that are received from the physician's EHR system 104 include a comment from the physician in addition to the medical results or just the comment. The medical results receive may also include one or more action items (discussed elsewhere in this document). At this step the patient-centric EHR system 102 may store the medical results (including any comments and/or action items) associated with the subscribing patient in a record associated with the subscribing patient.

At step 804, the patient-centric EHR system 102 then sends a notice to the subscribing patient's computing entity 106 associated with the subscribing patient that medical information is available. The notice that medical information is available may depend on the type of delivery method that the subscribing patient desires to obtain notices by. For example, the subscribing patient may provide an email address to receive the notice in the form of an email; a cell-phone number to receive the indication as a text message. In other cases, where the subscribing patient's computing entity 106 runs an application associated with the patient-centric EHR system 102, the application may provide the notice in the form of a pop-up box or window type notice or a red circle in a top corner of an icon associated with the application running on the subscribing patient's computing entity 106. Regardless of the means for providing the notice, the notice may only note that medical information is available for viewing at the patient-centric EHR system 102 and not provide any specific details regarding the medical information. (Step 804 of process 800 is similar to step 704 of process 700).

At step 806 a request for the medical information from the subscribing patient's computing entity 106 is received and the request may include authentication information. In the cases where the notice is provided via email or text-message, the subscribing patient via the subscribing patient's computing entity 106 may login to the patient-centric EHR system 102 to obtain the medical information. For instance, the subscribing patient may provide a user account identifier and a password. In the cases where the notice is provided in the form of a pop-up box or window type notice in the application running on the subscribing patient's computing entity 106, the subscribing patient may provide a user account identifier, a password and/or biometric authentication information. In other words, at this step the subscribing patient's computing entity 106 requests from the patient-centric EHR system 102 access to medical information by providing authentication information. (Step 806 of process 800 is similar to step 706 of process 700).

At step 808, the authentication information included in the request for the medical information is authenticated for example by comparing the authentication information against a record associated with the subscribing patient. (Step 808 of process 800 is similar to step 706 of process 700). Then, at step 810, if the authentication information is associated with the subscribing patient, the patient-centric EHR system 102 then transmits a notice to the subscribing patient's computing entity 106, the notice includes the medical information. In this case, the medical information is the medical results. If the medical results contain a comment from the physician, the comment from the physician regarding the medical results may also be included in this notice.

FIG. 16C illustrates a specific and non-limiting example where the patient via the patient's computing entity 106 may then view these notices. As illustrated, a first notice is provided that indicates that medical results have been received at the physician's office and a second notice is provided that includes the medical results and an annotation.

In some cases, step 804 may be omitted from the process 800. In these cases, a notice of the availability of medical information is not transmitted to the subscribing patient's computing entity 106 and the patient-centric EHR system 102 waits for a request from the subscribing patient's computing entity 106.

In other cases, step 808 may be omitted from the process 800 and prior to step 804 the subscribing patient's computing entity 106 provides authentication information and if the authentication information is associated with the subscribing patient, then a notice may be sent at step 804. In other words, notices are not sent unless the subscribing patient has authenticated his/her patient's computing entity 106 to the patient-centric EHR system 102 in a manner that indicates he/she is currently using the device and would like notices to be received.

In some embodiments, the medical results provided in the notice may be transmitted to the patient's computing entity 106 without any comment by the physician. In such case, the physician's EHR system 104 may automatically on creation of a medical record at the physician's EHR system 104 or on receipt of a medical record including medical results (e.g., from the EHR network 108), transmit the medical record including the medical results to the patient-centric EHR system 102. The patient-centric EHR system 102 may then notify the patient's computing entity 106 of the medical results in a similar fashion as discussed elsewhere in this document.

In other embodiments, the medical results transmitted to the patient's computing entity 106 may not contain the medical results received from the medical facility (e.g., lab, imaging facility, etc.) but includes only a comment or annotation from the physician. In other words, the medical results may not actually be transmitted by the physician's EHR system 104 and/or the patient-centric EHR system 102 and in these cases, annotations of the medical results may be provided instead. In such case, the process for transmitting the comment on the medical results to the patient's computing entity 106 from the physician's EHR system 104 via the patient-centric EHR system 102 may be similar to the process discussed elsewhere in this document in relation to transmitting the medical results to the patient's computing entity 106 from the physician's EHR system 104 via the patient-centric EHR system 102.

At step 810, the notice of the medical results associated with the subscribing patient is transmitted to the subscribing patient's computing entity 106 may also include an action item. In other words, the notice may be flagged as requiring patient interaction or not, as well as what type of action is required. For instance, the action item may be a follow-up appointment with the physician, a prescription prescribed by the physician or any other suitable action.

In the case that the action item is an appointment with the physician, the patient may be able to accept or reject an appointment provided via the notice that provided the patient's computing entity 106 with the medical results and the action item. In some cases, the appointment in the notice is a fixed time and date and the patient has the option of accepting or rejecting the time and date. Then the patient-centric EHR system 102 receives the acceptance or rejection of the appointment from the patient's computing entity 106. The patient-centric EHR system 102 may then store the acceptance or rejection in the records associated with the patient at the patient-centric EHR system 102. The patient-centric EHR system 102 may also transmit the acceptance or rejection of the appointment to the physician's EHR system 104 and in this case, the physician's EHR system 104 may then store the acceptance or rejection in the medical records associated with the patient and/or patient scheduling software. In other cases, the action item in the notice may provide for a means for the patient to schedule an appointment. For example, the action item may provide a link to the physician's website that allows the patient via the patient's computing entity 106 to book an appointment online. By way of another example, the action item may include an email address or phone number that the patient may then use to schedule an appointment.

In the case that the action item is a prescription, the action item may be provided in various forms. For example, the patient may be able to take the patient's computing entity 106 to a pharmacy and show the prescription to the pharmacy for the fulfillment of the prescription; the action item may indicate a pharmacy where the prescription may be picked-up; the action item may indicate that the prescription has been registered with the government regulated and/or state-owned or HMO EHR system and that a pharmacy that has access to this system may fulfill the prescription. Upon fulfillment of the prescription, the patient-centric EHR system 102, the EHR network 108 and/or the physician's EHR system 104 may receive acknowledgment of the fulfilled prescription. In other words, the patient-centric EHR system 102 and/or the physician's EHR system 104 may receive notification that an action item has been completed by the patient.

In some embodiments, patient-centric EHR system 102 receives a read-receipt from the patient's computing entity 106 after the patient via the patient's computing entity 106 views a notice (e.g., the medical results associated with the patient). The patient-centric EHR system 102 may then store the notice of the read-receipt in the records associated with the patient at the patient-centric EHR system 102. The patient-centric EHR system 102 may also transmit the read-receipt to the physician's EHR system 104 and in this case, the physician's EHR system 104 may then store the read-receipt in the medical records associated with the patient. It is appreciated that such a process may be used to inform the physician if the patient has received medical results, a comment associated with the results and/or any follow up actions.

The notice may be flagged by the patient-centric EHR system 104 with an expiry date-time stamp. If there is a request for the medical information associated with the notice after the time stamp, the request may be ignored by the patient-centric EHR system 104. In the case that the patient's computing entity 106 is running application software associated with the patient-centric EHR system 104, this software may remove notices that have expired. In other cases, the medical results provided in the notice may expire after a set period of time. The notices are discussed elsewhere in this document, such as the discussion in association with FIGS. 17A and 17B.

It is appreciated that the steps of process 700 and 800 may be considered a “pull” (or “client-pull”) based notification system instead of a “push” based notification system. To maintain confidentiality of the patient's medical results it is desirable for the medical results delivery system to be provided in a “pull” based fashion. If the patient's computing entity 106 is in the hands of a third-party, and the notification system is a “push” based notification system the confidentiality of the patient's medical results may be breached if the third-party views the notification. From a human perspective a “pull” can be made to look like a “push” but generally speaking is more desirable in terms of maintain confidentiality of the patient's medical results. For instance, when the patient-centric EHR system 102 receives a “pull” request, prior to sending the information requested, the patient-centric EHR system 102 verifies that the patient's computing entity 106 is verified to pull. Although in some embodiments, the patient via the patient's computing entity 106 initiates communication (e.g., a client-pull) with the patient-centric EHR system 102 to obtain various information (e.g., to get records, annotations, notices, at the like); in alternative embodiments, push notifications (e.g., email, push notification services, server-push systems, and/or any other suitable push notification) may be used. In other words, the only “push” that may be done in such a configuration is that a push of a communication indicating that there is some information available to be accessed without providing any details on the information available. Afterwards, a “pull” may be used to retrieve the available information once authentication has been done (e.g., verifying identity with a biometric reader).

The processes discussed above (e.g., the processes 700 and 800) provide for an identity management process. This identity management process allows for patients to receive communicates from the patient-centric EHR system 102 in a confidential way after the patient's identity has been authenticated. By providing a system where the patient is authenticate by the patient-centric EHR system 102 prior to communications possibly containing confidential information allows for a secure manner for communicating patent health related information.

It is also appreciated that after steps 710 or 810, that after the patient view the medical records and/or medical results on the patient's computing entity 106, that the patient's computing entity 106 may be configured to delete the medical records and/or medical results both in volatile and in persistent memory.

Although in the embodiment above, the physician's EHR system 104 provides the notices and medical results to the patient-centric EHR system 102 and it is the patient-centric EHR system 102 that provides the patient with the notices and medical results, in other embodiments, the physician's EHR system 104 may notify the patient directly without the use of the patient-centric EHR system 102. In other words, aspects of the patient-centric EHR system 102 may be incorporated into the physician's EHR system 104. As such, in some embodiment, some or all of the functionality of the patient-centric EHR system 102 discussed in this document may be implemented on the physician's EHR system 104.

In other embodiments, other forms of information exchange capability between the patient and the physician may be possible. In other words, the patient-centric EHR system 102 may provide for a means for facilitating the exchange of information between the physician via the physician's EHR system 104 and the patient's computing entity 106.

Notices

FIGS. 17A and 17B illustrate examples of a first notice 1700 and second notice 1800 in accordance with a specific and non-limiting example of implementation. As illustrated, the notices 1700 and 1800 include an identifier 1702, notice data 1704, an action 1708 and a timer 1710. The identifier 1702 is used to identify the patient's computing entity 106, such that the notice is communicated from the patient-centric EHR system 102 to patient's computing entity 106. The identifier 1702 may include an IP address, an email address, an account identifier, a telephone number and/or any other suitable identifier. The notice data 1704 may be used to communicate various information and data, such as: messages, medical results, annotations and/or any other suitable information. As illustrated, the notice data 1704 may include message data 1706, medical results data 1712, and/or an annotation data 1714. The message data 1706 may include a text-based and/or HTML based message used to provide information to the patient's computing entity 106. The medical results data 1712 may include the medical results and/or health records. The annotation data 1714 may be used to provide physician's annotation regarding the medical results. The action data 1708 may be used to convey an action item from the patient-centric EHR system 102 to the patient's computing entity 106. Such actions may include an allow/deny action, an acknowledge action (e.g., a delivery or read receipt action), an authentication action and/or any other suitable action.

It is appreciated that notices 1700 and 1800 are the general mechanism by with the patient associated with the patient's computing entity 106 becomes aware of any changes of his/her medical record. In fact, the first notice 1700 may also include a token (or a key), where the token may be used by the patient's computing entity 106 in making a pull to patient-centric EHR system 102 requesting further information and resulting in the patient's computing entity 106 receiving the second notice 1800 including the further information.

The notice 1700 is now discussed in the context of the example of the patient-centric EHR system 102 communicating a notice of authorization to the patient's computing entity 106. This notice of authorization typically is sent first, prior to sending any further notices, to authenticate that the person at the patient's computing entity 106 is in fact the patient. In this case, the message data 1706 may include a message that indicates to the patient that the patient-centric EHR system 102 has further information and/or a notice for him/her. The timer 1710 may be an expiry timer, for which the notice is set to expire.

In other words, the notice may no longer be accessible by the patient (e.g., the notice if removed from the notices application window on the application software running of the patient's computing entity 106) after a set period of time. In this example, the action 1708 includes an action item for the request for an identifier (e.g., a password, biometric information and/or any other suitable identifier) via the GUI of the patient's computing entity 106. The action item when acknowledged by the patient may send a communication from the patient's computing entity 106 to the patient-centric EHR system 102. In this case, the communication includes the identifier. The patient-centric EHR system 102 may process the received identifier to determine if the person at the patient's computing entity 106 is in fact the patient. This may be done by processing the received identifier against a record storing the patient's identifier. If the patient is authenticated, then further notices may be sent from the patient-centric EHR system 102 to the patient's computing entity 106. This may also be done by the patient-centric EHR system 102 communicating a token (or a key) in the first notice 1700 to the patient's computing entity 106 and then the validating the token in the request from the patient's computing entity 106 such that the patient-centric EHR system 102 verifies that the token is the token that was previously sent.

The notice 1700 is now discussed in the context of the example of the patient-centric EHR system 102 communicating a notice to get medical results done. In this example, the message data 1706 includes a message that indicates to the patient to go to a medical facility to get medical results done. The action 1708 in this example includes an action item for the patient to acknowledge via the GUI of the patient's computing entity 106 that he/she went to the medical facility to get the testing/lab-work done. The timer 1710 may be a reminder to the patient to get the medical testing or lab work done or may be an expiry timer for which the notice is set to expire and the patient can no longer get medical testing/lab-work done. For instance, if the patient does not go to get the testing/lab-work done after a set period of time, the patient-centric EHR system 102 may issue another notice and/or a reminder. Also, if the patient does not go and get the testing/lab-work done after a set period of time, the notice may no longer be accessible by the patient (e.g., the notice if removed from the notices application window on the application software running of the patient's computing entity 106).

The action item when acknowledged by the patient may send a communication from the patient's computing entity 106 to the patient-centric EHR system 102. The patient-centric EHR system 102 may process the receipt of an action item to setup other notices and/or reminders. For example, after the patient acknowledges that he/she got the testing/lab-work done, then the patient-centric EHR system 102 may process this acknowledgement against the type of testing/lab-work done to determine the typical timeframe for the medical results for this testing/lab-work. Then, if the patient-centric EHR system 102 does not receive acknowledgement from the physician's EHR systems 104 that the medical results have been received at the physician's office, the patient-centric EHR system 102 may issue a notice to the patient's computing entity 106 and/or physician's EHR systems 104 that the medical results were not received.

For example, if a patient gets a blood count test done, this typically only takes a couple of hours and if the patient via the patient's computing entity 106 has not received a notice that the test results are done within a 24 to 48 hour period, this may be an indication that the test results were lost or the blood work was not done. In other words, the patient-centric EHR system 102 may be able to determine if test/lab results are missing or not completed.

Turning now to FIG. 17C, a notice 1900, which is similar to the notices 1700 and 1800 is shown; however, the notice 1900 may be a communication that is made from the medical EHR system 110 to the physician's EHR system 104, either directly or via the EHR network 108. The notice 1900 may be a communication that is sent from medical EHR system 110, where the notice data 1704 include medical result associated with a patient. The notice 1900 may include an urgency indicator to indicate that the medical results are time sensitive and should be reviewed by the physician urgently. The incoming notices at the physician's EHR system 104 may be processed and when a notice includes an urgency indicator, it may be then brought to attention to physician via the physician's EHR system 104 in a more urgent manner than non-urgent notices. For example, after the patient visits the lab and the lab technician enters in the medical results from the patient's lab tests, the notice 1900 may be communicated from the medical EHR system 110 to the physician's EHR system 104, and in this example the medial results include an outcome/observation that requires urgent review by the attending physician or the primary physician of the patient, as the results of the test indicate an outcome that could have immediate affect on the patient's health condition. The timer 1700 of the notice 1900 may include a “time-out”, so that the issuing lab technician associated with the medical EHR system 110 is notified via the action 1708 which results in the physician's EHR system 104 sending a notice to the medical EHR system 110 that the physician has not seen the medical results within a specific amount of time.

Referring back to FIG. 17A, the notice 1700 is now discussed in the context of the example of the patient-centric EHR system 102 communicating a first notice of the availability of medical information to the patient's computing entity 106. In this case, the message data 1706 includes a message that indicates to the patient that his/her medical results have arrived at the physician's office. The message data 1706 may also include an indication that a notice will be provided later. The timer 1710 may be used to as reminder so the patient can follow-up with his/her physician if he/she does not receive a notice with the medical results in a set period of time. The timer 1710 may also be an expiry timer for which the notice is set to expire and the patient can no longer view this notice.

The notice 1800 is now discussed in the context of the example of the patient-centric EHR system 102 communicating a second notice of the availability of medical information to the patient's computing entity 106. In this case, the medical results data 1712 conveys the medical results and the annotation data 1714 includes a physician's comment or annotation regarding the medical results. The action 1708 may be used for various actions items such as acknowledgment of receipt of the notice, to schedule an appointment and/or to provide a prescription. The timer 1710 may be an expiry timer for which the notice is set to expire and the patient can no longer view this notice.

The physician should report the medical results to the patient in a specific time-frame and the patient-centric EHR system 102 may be able to record or track the time-frame that it takes each physician to report the medical results. In other words, the patient-centric EHR system 102 may monitor and record the time frames of the various timers and actions to produce reports that indicate the average medical facility processing time, the physician's time to review the medical results and/or any other suitable report. This data may be useful in determine which medical facilities process results faster than others and/or to determine which physicians review medical results faster than others.

The message data 1706, medical results data 1712 and the annotation data 1714 may specific that the notice or the information provided in the notice is “for your eyes only” (or a similar word having the same connotation), as it is appreciated that the patient may be receiving medical results and/or information that is sensitive in nature.

Notice 1700 and/or notice 1800 may also be used in various other embodiments to obtain permission for an action from the patient via the patient's computing entity 106. For example, to receive authorization from the patient to provide health records to a third-party, to receive authorization from the patient to have his/her health records used in a study, and/or to receive authorization from the patient any other suitable action.

In some specific examples of implementation the notices may include a must acknowledge field, an acknowledged by field and an expiry field. The must acknowledge field requires a receipt from the patient's computing entity 106. The acknowledged by field identifies who acknowledged and may include an identifier of the party that acknowledged the receipt of the notice. The expiry field is used to set an expiry timer.

This notification system may be useful when multiple test/lab results are being done, as it may allow for a means to monitor multiple test/lab results.

It is appreciated that timers may be used in various ways in the various embodiments described herein. In general any communication or notice (e.g., the notices 1700, 1800 and 1900) may include a timer 1710. Although the timer 1710 is discussed herein in various examples, more generally a timer may be used to notify patients, physician's, practitioners, and/or any other suitable person of an action that is required by him/her (e.g., view the notice, view the information contained in the notice, follow the action in the notice), any missed actions (e.g., a reminder), may be used for notices to become expired and no long visible by the user.

A delegate, such as a patient's legal guardian and/or any other suitable person, having a delegate's computing entity (similar to the patient's computing entity 106) may be setup for receiving notices with the patient-centric EHR system 102. The delegate's computing entity may receive notices instead of the patient's computing entity 106 or the patient's computing entity 106 and the delegate's computing entity may both receive notices. This may be useful where the patient is disabled or has limited mobility and needs regular assistance from another person.

Health Related Determination Mechanism

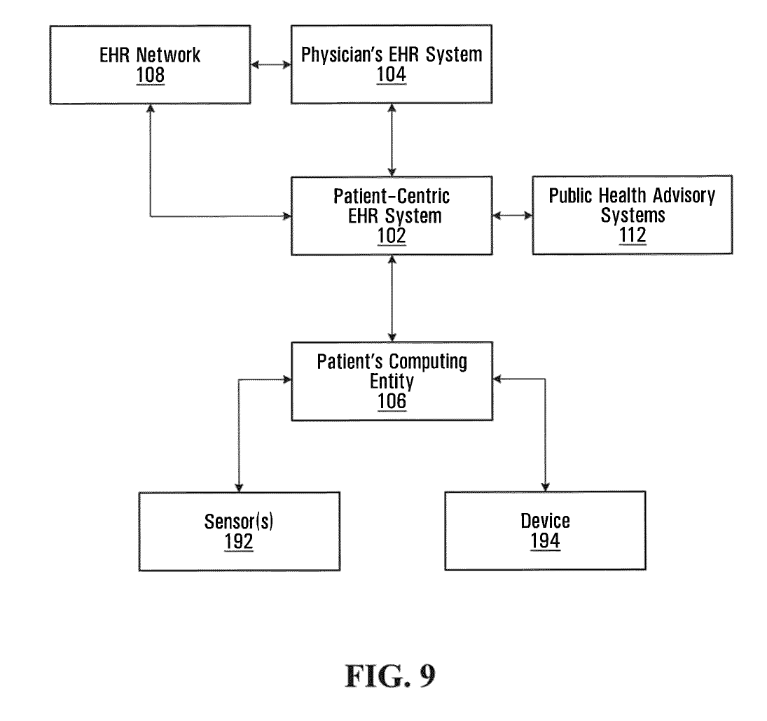

FIG. 9 illustrates a block diagram of an example of the patient-centric EHR system 102, where the patient-centric EHR system 102 is able to receive and obtain auxiliary medical related information. The patient-centric EHR system 102 may be connected to the patient computing entity 106, to the physician's EHR systems 104, to the EHR network 108 and/or to one or more systems that provide public health advisories 112. As illustrated, the patient computing entity 106 is connected to one or more sensors 192. In some cases, the patient computing entity 106 is also connected to an external device 194. The various connections between the patient-centric EHR system 102, the patient's computing entity 106, the physician's EHR system 104, the medical EHR system 110 (not illustrated in FIG. 9), the EHR network 108, the public health advisor systems 112, one or more sensors 192 and/or the external device 194 may include one or more connections over one or more data networks. The various connections between the patient-centric EHR system 102, the patient's computing entity 106, the physician's EHR system 104, the medical EHR system 110, the EHR network 108, the public health advisor systems 112, one or more sensors 192 and/or the external device 194 may allow for the communication (e.g., transmission and/or reception) of various information and/or data between the various systems and/or devices. The various information and/or data exchanged may be a “notice”, “notices”, “health record” and/or “health records” of the type discussed elsewhere in this document. In some embodiments the connection between the one or more sensors 192 and/or the external device 194 with the patient's computing entity 106 may be a wired (e.g., USB, USB-C, Ethernet, Firewire™ or any other suitable connection) or wireless connection (e.g., Bluetooth™, ZigBee™, Wi-Fi™, or any other suitable connection).

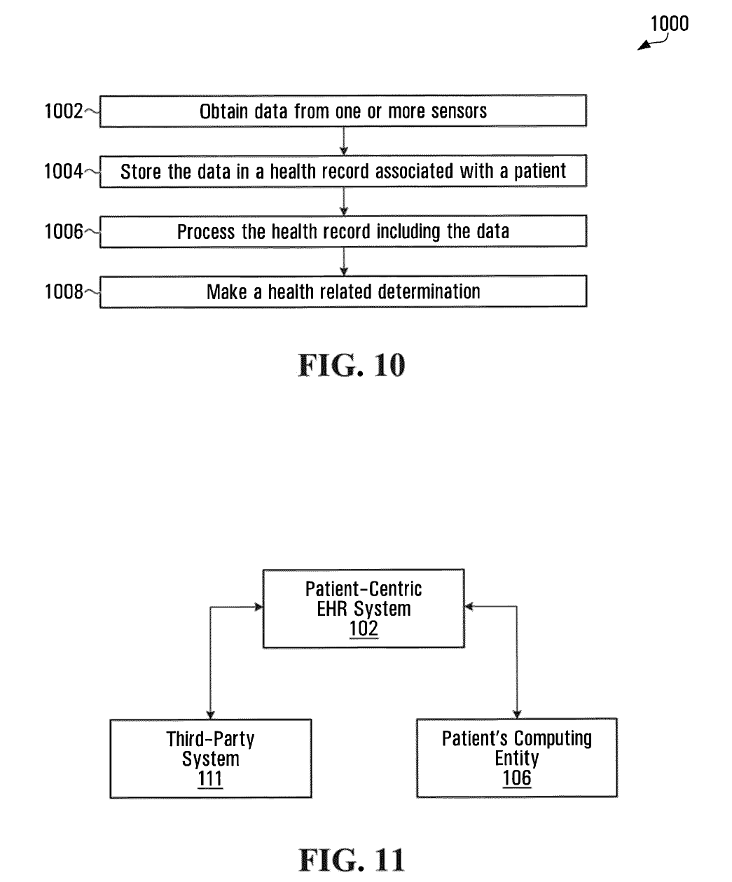

FIG. 10 illustrates a flowchart of a process 1000 which may be implemented at the patient-centric EHR system 102 for making a health related determination at least in part by processing data obtained from one or more sensors 192 in combination with health records associated with a patient. At step 1002, data from one or more sensors 192 is obtained. This may include monitoring data obtained from the one or more sensors 192 where these sensors 192 measure a physical condition of a patient and where the monitoring of the data from the one or more sensors is done over a plurality of time points. These one or more sensors 1002 may be of various types, including: blood pressure monitors, blood glucose monitors, heart rate monitors, accelerometers, GPSs, barometer, altimeter, ambient light level detector, spectrum analyzer, any other sensors located in a portable computing device (e.g., smart-watch, portable phone, tablet, PDA, or any other suitable portable computing device), and/or any other suitable sensor or computing device. At this step, data from public health advisory systems 112 may also be obtained, which may include various types of information such as smog warnings, heatwaves and/or any other suitable information.

At step 1004 the data from the one or more sensors 192 is stored in association with a health record associated with a patient. It is appreciated that the data from the one or more sensors may be collected over multiple time points to form longitudinal data. This longitudinal data associated with the patient may track a physical condition of a patient over a period of time. This longitudinal data may also include environmental factors such as atmospheric pressure, UV exposure and/or any other suitable environmental factors. This longitudinal data associated with the patient may be stored in one or more health records on the patient-centric EHR system 102. The patient-centric EHR system 102 may also contain a continuum of heath records associated with the patient obtained from various EHR systems and/or the EHR network 108. In other words, the medical records may have been obtained from a plurality of EHR systems and/or other sources. This longitudinal data in combination with the continuum of heath records associated with the patient may allow for processing of this information to make a health related determination for the patient, such as may be done at step 1006. In general, at step 1006, health records including the data from the sensors 192 are processed. This may include processing the data from the sensors 192 against the medical records associated with the patient or processing the health records associated with the patient where these health records are augmented by the data from the sensors 192. This processing may also include tracking trends in the longitudinal data. Then at step 1008, at least in part by processing the health record including the data from the sensors a health related determination is made. The health related determination may be made when a threshold level is detected. The detection of the threshold level being reached may be determined by computer processing. The detection of the threshold level being reached may also be verified by a third-party agent (e.g., a person). The health related determination may include sending a notice to the patient via the patient's computing entity 106. The health related determination may include a notice being sent to the patient's primary physician, assuming that the patient has named a primary physician. In the case that a third-party agent verifies the threshold level being reached the agent may initiate the communication of the notice to the patient and/or to the physician to indicate the unsafe condition. In the case that the notice from a heath related determination is sent to the physician's EHR system 104, the physician may then be notified via the physician's EHR system 104 to review the notice and then make a determination to notify the patient, which may then be sent from the physician's EHR system 104 to the patient's computing entity 106 via the patient-centric EHR system 102.

The process 1000 should likely become more readily apparent in view of the following examples:
Blood pressure monitor example: In this example the sensor 192 is a sensor that monitors blood pressure. The sensor 192 is in communication with the patient's computing entity 106 and the patient's computing entity 106 communicates blood pressure data to the patient-centric EHR system 102, such that the patient-centric EHR system 102 may monitor this data. The patient's health records indicate that the patient has high blood pressure and the patient-centric EHR system 102 based on processing the patient's health records determines that if the blood pressure of the patient rise above a certain threshold that the patient via the patient's computing entity 106 and/or the patient's physician via the physician's EHR system 102 should be notified as this may be an indication of a possible medical issue.
Blood glucose monitor example: In this example the sensor 192 is a sensor that monitors blood glucose levels. Various blood glucose monitors are currently available in the market (e.g., MyGlucoHealth Blood Glucose Meter with Bluetooth Technology). The sensor 192 is in communication with the patient's computing entity 106 and the patient's computing entity 106 communicates blood glucose data to the patient-centric EHR system 102, such that the patient-centric EHR system 102 may monitor this data. The patient's health records indicate that the patient has diabetes and the patient-centric EHR system 102 based on processing the patient's health records determines that if the blood glucose level of the patient raises above a certain threshold that the patient via the patient's computing entity 106 and/or the patient's physician via the physician's EHR system 102 should be notified as this may be an indication of a possible medical issue.
Heart rate monitors & exercise equipment example: In this example the sensor 192 is a sensor that monitors heart rate. Various heart rate monitors are currently available (e.g., OMsignal Biometric Smartwear). The sensor 192 is in communication with the patient's computing entity 106 and the patient's computing entity 106 communicates heart rate data to the patient-centric EHR system 102, such that the patient-centric EHR system 102 may monitor this data. In this example, the device 194 generally speaking is a piece of exercise equipment and in particular is a treadmill. Prior to starting to run on the treadmill the patient identifies the sensor 192 and device 194 to the patient-centric EHR system 102. The patient-centric EHR system 102 based on the patient's health records make a determination that the patient should run at a specific pace and may control the speed of the treadmill. As the patient starts to run on the treadmill, his/her heart rate increase and in this example the heart rate is too high and the patient-centric EHR system 102 makes the determination by processing the heart rate data in combination with the patient's health records that the speed of the treadmill should be reduced. In other words, in this example, the patient-centric EHR system 102 may make a recommendation of a treadmill speed based on the patient's health records and may adjust the speed in accordance with changes in heart rate.
Chronic obstructive pulmonary disease, GPS & public health advisory example: In this example the sensor 192 is a GPS sensor which may be part of the patient's computing entity 106. The sensor 192 is in communication with the patient's computing entity 106 and the patient's computing entity 106 communicates GPS location data to the patient-centric EHR system 102, such that the patient-centric EHR system 102 may monitor this data. The patient's health records indicate that the patient has chronic obstructive pulmonary disease (COPD) and the patient-centric EHR system 102 based on processing the patient's health record determines that the patient-centric system should obtain public health advisories for the public health advisory system 112. Then the patient-centric EHR system 102 monitors the location of the patient and processes the public health advisories to determine if the patient is in a high smog area and if such is the case, notifies the patient via the patient's computing entity 106 and/or the patient's physician via the physician's EHR system 102.
The above examples are intended to be non-limiting and various other examples of using the sensors 192 in combination with the patient's health records obtained for multiple EHR system and/or sources to make health related determinations are possible.

The patient-centric EHR system may be provided with logic that can process the information received from the various biometric sensors and generate calibration data sent to wearable or stationary devices intended to provide guidance to the patient regarding physical activity. For example, the program logic can interpret the physiological information received such as to set a safe level of activity that the user should not exceed in order to avoid a health risk, such as a heart attack, stroke or other. To be more specific, the program logic will process the physiological information such as the hear rate, blood pressure and blood glucose, among others to derive the safe level for physical activities. This processing can be performed by mapping the physiological data with predetermined degrees of “safe level” exercise intensity, such as exercise duration, speed of running, maximal heart level rate during the exercise, etc. In a possible refinement, the program logic can use other information from the patient record, which is co-related to the physiological information to further refine the “safe level” for the user based on the user's medical history.

The calibration data can be sent to the physiological sensor that generated the physiological data when that sensor is used to provide physical activity guidance to the user, such as the degree of daily physical activity desired. The calibration data will thus determine the amount of activity (calories burned) and the intensity.

In one specific example, the calibration data can be sent to an exercise machine, such as a treadmill, to set the operational parameters of the treadmill for an exercise session for the user, such as the maximal running speed, duration, elevation, etc. A similar approach can be considered for another kind of exercise equipment such a muscle building machine.

In other embodiments instead of the patient-centric EHR system 102 processing the health record associated with the patient and the auxiliary medical related information (e.g., data from sensors 192, device 194, public health advisory system 112, and/or any other suitable information) the patient-centric EHR system 102 may transmit the health record associated with the patient and the auxiliary medical related information to a third-party system (e.g., an agent) which may then process the health record and auxiliary medical related information to make the health related determination in a similar fashion as that discussed above. In such case, after the third-party system makes the health related determination, the third-party system may transmit the health related determination to patient-centric EHR system 102 which may forward the health related determination to the physician's EHR system 104 and/or the patient's computing entity 106.

It is appreciated that the health related determination may be made by a computer based decision support agent that contains logic that can make a health related determination. More specifically, the decision support agent may process the health record associated with the patient and the auxiliary medical related information in relation to rules, fact, fact and rules, models, algorithms, search, procedural code, analytic techniques, inference engine and/or any other suitable technique or logic to make the health related determination that may add value to the patient. In some cases the decision support agent is an artificial intelligence and/or expert system that may emulate the decision making ability of a human expert.

Although in the examples above the health record associated with the patient and the auxiliary medical related information was processed to make a health related determination, in other embodiments the health record associated with the patient may be processed on its own (without the auxiliary medical related information) to make a health related determination in a manner similar to that discussed above.

Third-Party Medical Record Access Mechanism

FIG. 11 illustrates a block diagram of an example of the patient-centric EHR system 102, where the patient-centric EHR system 102 is able to provide a health record associated with a patient to a third-party system 111 upon authorization of the patient at the patient computing entity 106 (e.g., a computing entity associated with the patient) in accordance with an embodiment of the invention. The third-party system 111 may be an EHR system similar to the physician's EHR system 104, discussed elsewhere in this document. As illustrates the patient-centric EHR system 102 is connected to the patient's computing entity 106 and to a third-party systems 111 (e.g., an system associated with a third-party). The various connections between the patient-centric EHR system 102, the patient's computing entity 106 and/or the third party system 111 may include one or more connections over one or more data networks. The various connections between the patient-centric EHR system 102, the patient's computing entity 106 and/or the third party system 111 may allow for the communication (e.g., transmission and/or reception) of various information and/or data between the various systems and/or devices. The various information and/or data exchanged may be a “notice”, “notices”, a “health record” and/or “health records” of the type discussed elsewhere in this document. The third party system 111 may be viewed as an agent. For example, it may be a system that examines records associated with the patient. The agent may be able to examine or process the records and make various determinations and/or annotations to one or more of the records. It is appreciated that the agent may be a person at the third party system 111 that reviews the views the health records and then runs additional tests or processes on the health record to make the various determinations and/or annotations to one or more of the records. In some embodiments, the agent may be a computer based agent comprising the computer based decision support agent (discussed elsewhere in this document) that may process the health records and make various determinations and/or annotations to one or more of the records.

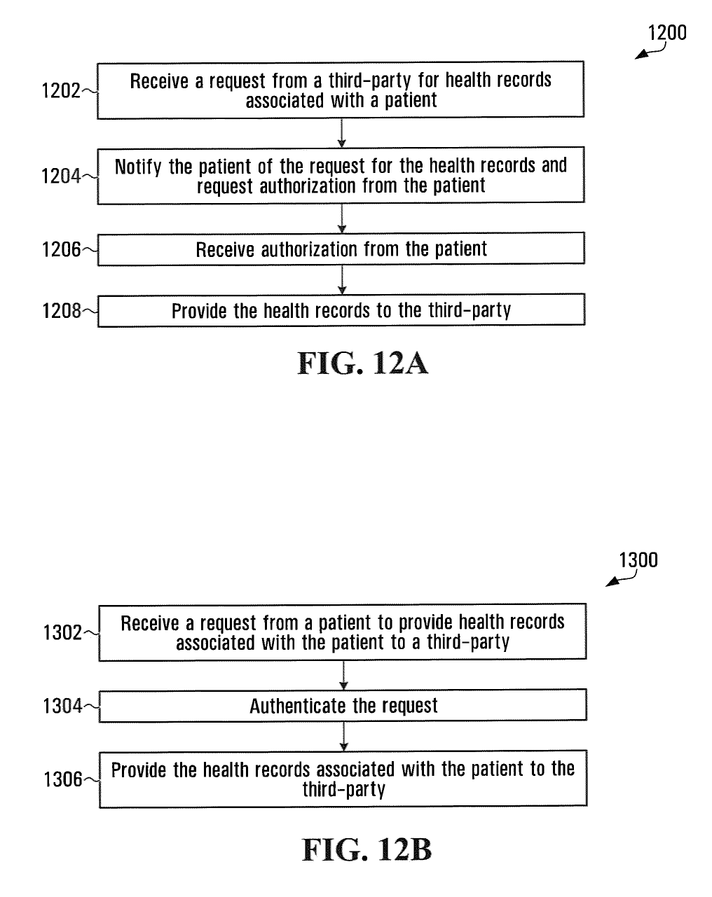

FIG. 12A illustrates a flowchart of a first process 1200 for providing one or more health records associated with a patient to the third-party system 111. At step 1202 a request from a third-party system 111 for one or more health records associated with a patient is received at the patient-centric EHR system 102. The request may be initiated from the third-party system 111. At step 1204, the patient associated with the heath record which was requested is notified of the request for the one or more health records via the patient's computing entity 106. The patient-centric EHR system may also request that the patient via the patient's computing entity 106 provide authorization to provide the third-party system 111 with the one or more health records. In this example, the patient's computing entity 106 is provided with the notice (similar to the notices discussed elsewhere in this document) and the request for authorization (e.g., an action item). At step 1206, the patient-centric EHR system 102 receives the authorization from the patient via the patient's computing entity 106. Then, at step 1208, the one or more health records associated with the patient are provided by the patient-centric EHR system 102 to the third-party via the third-party system 111. If the authorization of the patient at step 1206 fails or the patient declines to authorize the patient-centric EHR system 102 to provide the records to the third-party system 111, the process 1200 would stop (i.e., no records would be provided at step 1208).

Steps 1204 may be similar to step 704 discussed above, in that a notice is sent to the patient via the patient's computing entity 106. The notice may not provide any indication of the contents of the notice other than indicating that some action/information is available. The patient may then authenticated himself/herself at step 1206 (e.g., similar to step 706) and the patient-centric EHR system 102 would then authenticate the request (e.g., similar to step 708). Once the patient's computing entity 106 is authenticated to the patient-centric EHR system 102 a common proxy identifier may be used to communicate data between the patient's computing entity 106 and the patient-centric EHR system 102. Then the patient's computing entity 106 may send the request to the patient-centric EHR system 102 to authorize the patient's computing entity 106 to provide one or more records associated with the patient to the third-party system 111.

FIG. 12B illustrates a flowchart of a second process 1300 for providing one or more health records associated with a patient to a third-party. The process 1300 is similar to process 1200; however, the process 1300 may be initiated by the patient while the process 1200 may be initiated by the third-party. At step 1302 a request from a patient via the patient's computing entity 106 is received at the patient-centric EHR system 102. The request is for the patient-centric EHR system 102 to provide a health record associated with the patient to a third party. This request from the patient may occur after the patient has authenticated himself/herself to patient-centric EHR system 102, as discussed above. In this example, the third-party is associated with the third-party system 111. At step 1304, the request is authenticated and then, at step 1306, the one or more health records associated with the patient are provided to the third-party system 111. Step 1304 and 1306 are similar to steps 1206 and 1208 discussed above. If the authorization of the patient at step 1304 fails, the process 1300 would stop (i.e., no records would be provided at step 1306).

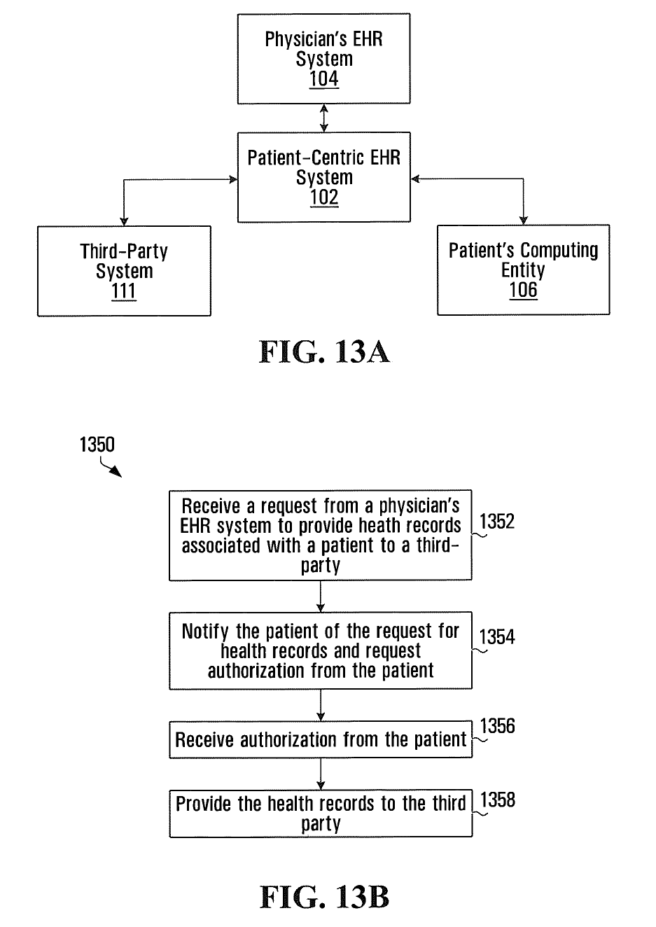

FIG. 13B illustrates a flowchart of a third process 1350 for providing one or more health records associated with the patient to the third-party system 111. The process 1350 is similar to that of the process 1200; however, the request to provide the one or more health records associated with the patient to the third-party system 111 is initiated by the physician at the physician's EHR system 104. For instance, the process 1350 may be implement on the patient-centric EHR system 102 shown in FIG. 13A, which illustrates a block diagram of an example of the patient-centric EHR system 102 shown in FIG. 11, but where the physician's EHR system 104 is in communication with the patient-centric EHR system 102 for initiating the request to provide the one or more health records associated with the patient to the third-party system 111. Turning back to the process 1350, at step 1352 a request from the physician's EHR system 104 to provide one or more health records associated with a patient to the third-party system 111 is received at the patient-centric EHR system 102. At step 1354, the patient associated with the heath record which was requested is notified of the request for the one or more health records via the patient's computing entity 106. The patient-centric EHR system may also request that the patient via patient's computing entity 106 provide authorization to provide the third-party system 111 with the one or more health records. Step 1354 may be implemented in a similar way as step 1204 discussed above. At step 1356, the patient-centric EHR system 102 receives the authorization from the patient via the patient's computing entity 106. Step 1356 may be implemented in a similar way as step 1206 discussed above. Then, at step 1358, the one or more health records associated with the patient are provided by the patient-centric EHR system 102 to the third-party system 111. Step 1358 may be implemented in a similar way as step 1208 discussed above. If the authorization of the patient at step 1356 fails or the patient declines to authorize the patient-centric EHR system 102 to provide the records to the third-party system 111, the process 1350 would stop (i.e., no records would be provided at step 1358).

Also, although not illustrated in FIGS. 12A, 12B and 13B, the patient may at a later time withdraw authorization to the third-party to have access to the health records associated with the patient. For instance, the patient via the patient's computing entity 106 may authenticate himself/herself to the patient-centric EHR system 102 (as discussed elsewhere in this document), afterwards the patient via the patient's computing entity 106 may send a request to the patient-centric EHR system 102 to remove or revoke access to a third-party to one or more of the health records that the third-party previously had access thereto. In other words, the patient with the patient's computing entity 108 may connect to the patient-centric EHR system 102 to control who has visibility access to his/her entire health record or to specific records.

The processes 1200,1300 and 1350 are illustrated further by way of a specific and non-limiting example. In this example, the patient-centric EHR system 102 has an agent company providing ‘on-call’ physicians that can read one or more health records and provide interpretation/information. The patient may first subscribe to the agent and then the patient may request a second opinion on one or more health records (e.g., such as at step 1302). For instance, the patient may subscribe directly to the third-party system 111 or may subscribe to the third-party system 111 via the patient-centric EHR system 102. The third-party system 111 is notified via the patient-centric EHR system 102, assigns a third-party physician, and issues a notice to authorize (e.g., patient-physician pairing) via the patient-centric EHR system 102. The patient receives the notice (e.g., such as at step 1204). The notice includes an action which requires confirmation and the patient confirms (e.g., such as step 1206). The third-party physician via the third-party system 111 receives a notice with the one or more health records attached (e.g., such as at step 1208 or 1306). For example, the third-party physician may connect to the third-party system 111 and/or to the patient-centric EHR system 102 via the third-party system 111 to gain access to the patient's health records. Then the third-party physician may add a determination and/or an annotation to the one or more health records. When a determination and/or annotation is added to the one or more health record associated with the patient on the patient-centric EHR system 102, the patient at the patient's computing entity 106 may receive notice from the patient-centric EHR system 102, with the attached third-party physician's determination and/or annotation. The primary physician, of the patient, via the physician's EHR system 104 may also be notified when a determination and/or an annotation is added to a health record associated with the patient.

Similarly, the example above instead of the health records associated with the patient being provided to a physician for review, the health records may be provided to a third-party system 111 for processing the patient's records to make a health related determination. For example, the determination may be to identify some genetic predisposition that the patient has and/or any other suitable medical condition. In other words, the patient may provide at least some of his/her health records to the third-party system 111 for processing or continuous monitoring, such that the system may make a health related determination and then provide such heath related determination to the patient. For instance, the health related determination may be made by the computer based decision support agent that contains logic that can make a health related determination, discussed elsewhere in this document. The computer based decision support agent may also be used for continuous monitoring of the patient's health record and when a possible medical condition is detected, cause a notification to be sent to the patient and/or the patient's physician.

The processes 1200,1300 and 1350 are illustrated further by way of a specific and non-limiting example. In this example, the patient is visiting family in Australia and the patient has a care need. The patient finds a third-party physician and would like to provide this third-party physician with his health records. The patient via the patient's computing entity 106 grants the third-party physician/the third-party physician's clinic access rights to his health records on the patient-centric EHR system 102. The patient via the patient's computing entity 106 may connect or log into the patient-centric EHR system 102 and select the third-party that the patient desires to share his health records with (e.g., such as at step 1302), as is shown in example illustrated in FIG. 16F. The patient may be able to set a start and end date (and time) or an expiry date (and time) for which the third-party physician can have access to the patient's health records. The patient may also be able to select (e.g., flag) which health records that the patient would like to have provided to the third-party physician. After the patient-centric EHR system 102 receives this request from the patient to grant the third-party physician access rights to the selected health records, the patient-centric EHR system 102 notifies the third-party physician via the third-party system 111. Although the third-party system 111 in some embodiments is an EHR system, in other cases this system 111 may not have EHR capabilities but may be any portable or non-portable computing device and/or entity, such as a cell-phone, a tablet, a smart watch, a laptop/notebook computer, a computer or any other suitable computing device. In this example the third-party system 111 associated with the third-party physician receives a notice from the patient-centric EHR system 102. This notice may be in the form of a secure email granting the third-party physician access to the patient-centric EHR system 102 to access the selected health records associated with the patient. In this example, the third-party physician's access to the patient-centric EHR system 102 would be limited to the health records associated with that patient only and may be limited to health records that the patient chooses to share with the third-party physician. The third-party physician logs-in with his third-party computing device 111 and has access to whatever parts the patient has selected as accessible by the third-party physician. As such, the patient is able to provide access right to all of his health records or to select with health records that the patient would like to grant access to. In this example, before the third-party physician can actually see the health records associated with the patient, the patient's computing entity 106 receives notice with an allow or deny confirmation option (e.g., such as at step 1204). This option allows for the patient to deny access, for example if the patient is not in the presence of the third-party physician. If the patient allows access (e.g., such as at step 1206), for example, when the patient has the benefit of visually verifying the identity of the third-party physician, the third-party computing device 111 is then provided with the health records associate with the patient that the patient has selected to be accessible by the third-party physician. In this example, the patient may use biometric authentication (or any other suitable means of authentication) on the patient's computing entity 106 in order to confirm that the actual patient, as opposed to the person who has the device in hand, is authorizing the access.

Care Support Group

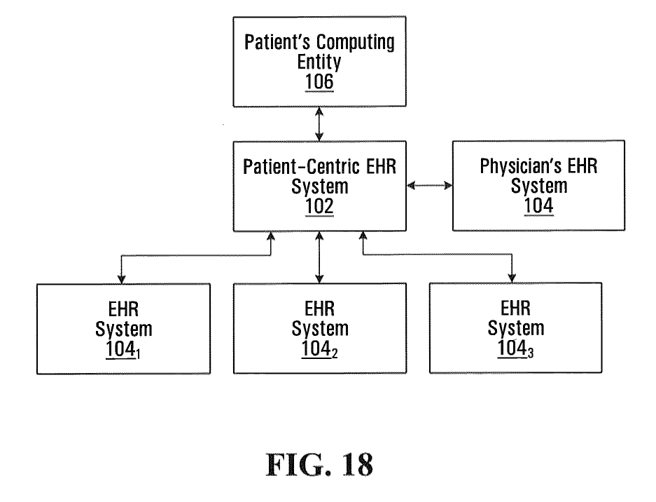

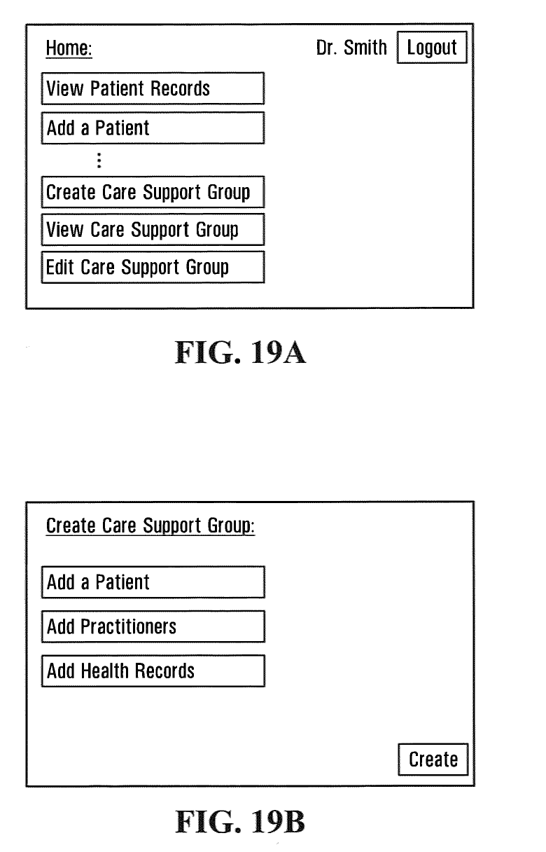

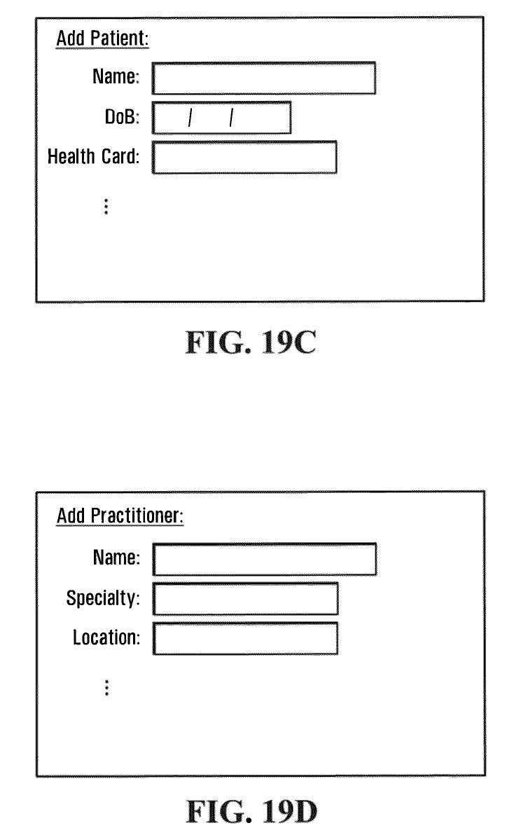

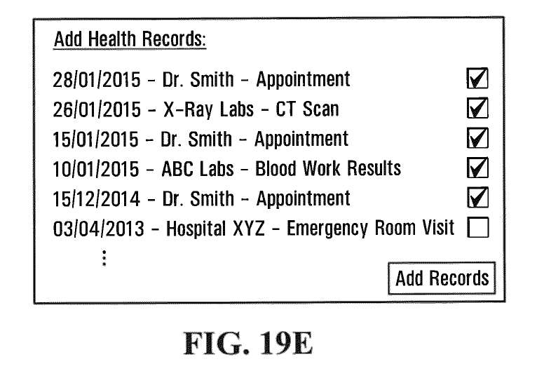

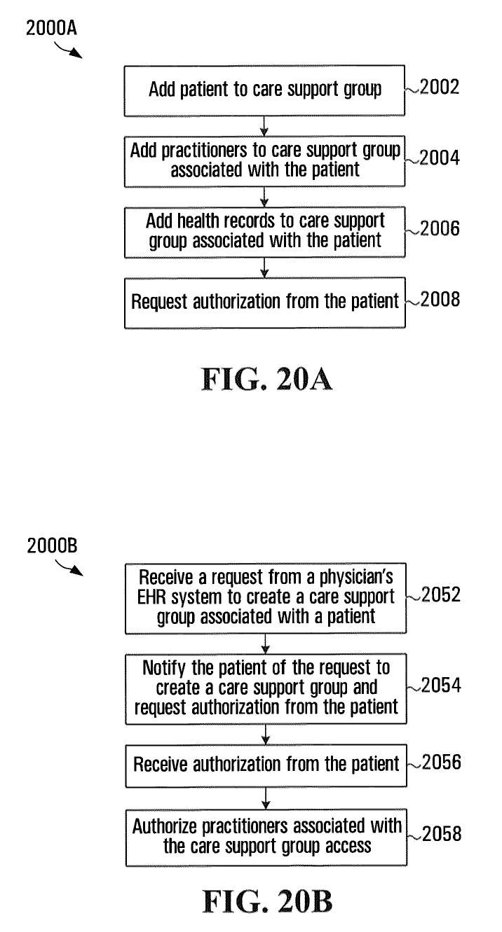

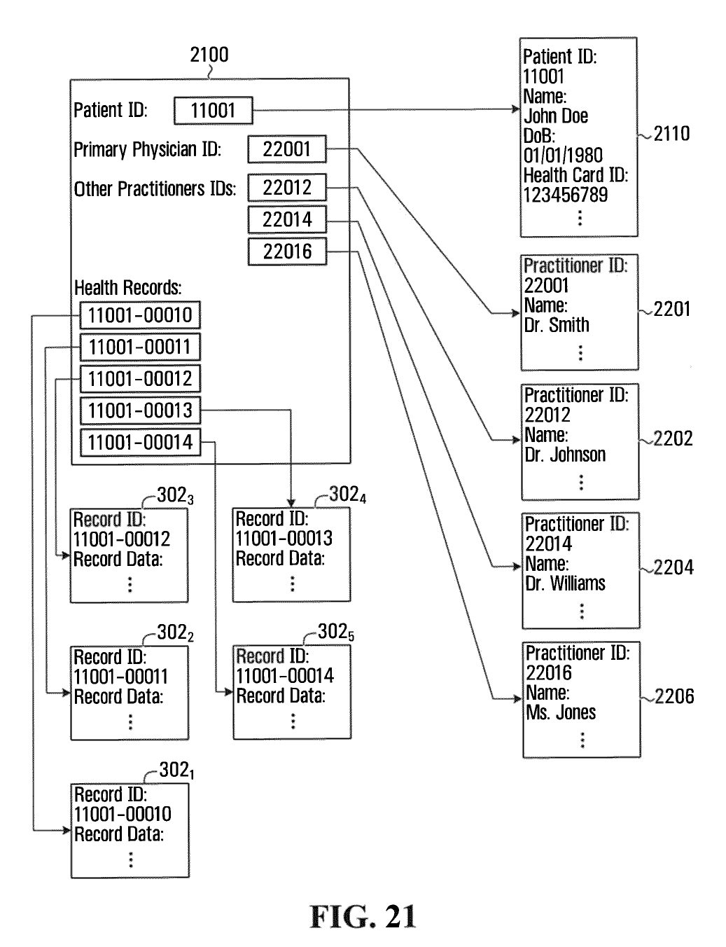

A care support group system will now be described with reference to FIGS. 18, 19A to 19E, 20A, 20B and 21. In general, the care support group may be considered a subset of the health records associated with a patient that is accessible and updatable by a select group of practitioners. FIG. 18 illustrates a block diagram of an example of the patient-centric EHR system 102 connected to a primary physician's EHR system 104, a plurality of EHR systems 104₁ 104₂ 104₃ associated with other practitioners and to the patient's computing entity 106 in accordance with an embodiment of the invention. In this embodiment, the primary physician via the physician's EHR system 104 is able to create a care support group around a patient and a situation associated with the patient via the patient-centric EHR system 102. FIGS. 19A to 19E illustrate specific and non-limiting examples of information that may be displayed on a graphical user interface (GUI) of a physician's computing entity in the process of creating the care support group in accordance with specific and non-limiting examples of implementation. FIGS. 20A and 20B illustrate flowcharts of processes 2000A and 2000B for creating the care support group in accordance with a specific non-limiting example of implementation. FIG. 21 illustrates an example of a computer readable data structure 2100 for the care support group that may be stored on the patient-centric EHR system 102 in accordance with a specific non-limiting example of implementation.

It is appreciated that the primary physician associated with the patient may want to share various health records and information associated with a patient in order to provide care to the patient. For instance, the primary physician may want to create a therapeutic team for the collaboration in the treatment of a specific condition (e.g., condition, disorder, infection, disability, injury, situation and/or any other suitable condition or situation) associated with the patient. For instance, the medical condition may have specific symptoms and signs that the physician has identified which may be determined based on testing results (e.g., blood tests, urine tests, x-ray images, and/or any other suitable test). As such, the physician may want to further investigate and/or treat the medical condition. Regardless of the specific condition or situation for creating the care support group around, the physician may interact with the patient-centric EHR system 102 to create the care support group further described below.

A specific and non-limiting example of the care support group (or circle of care) will now be described. In this example, the patient John Doe was diagnosed as having cancer by his primary physician Dr. Smith. As John Doe chooses to undergo various treatments based on the advice of his primary physician, Dr. Smith chooses to interact with the patient-centric EHR system 102 via his physician's EHR system 104 to create a care support group during the treatment of John Doe's cancer. As such, the primary physician Dr. Smith connects to the patient-centric EHR system 102 which may include him logging in with the physician's EHR system 104 to the patient-centric EHR system 102 by providing his account identifier and credential. After logging in to the patient-centric EHR system 102, the GUI of the display device associated with the primary physician's EHR system 104 may be conditioned to display the information shown in FIG. 19A. As shown, FIG. 19A provides Dr. Smith with various interface controls to allow him to select and view health records associated with a patient, add a patient to the patient-centric EHR system 102, create a care support group, view a care support group and edit a care support group. Other interface controls may be available in other embodiments.

The primary physician Dr. Smith may select the interface control to create a new care support group and then may be provided with the information shown in FIG. 19B, which includes interface controls for adding a patient, adding practitioners. Other interface controls may be included in other embodiments. Afterwards, the primary physician Dr. Smith may create the care support group according to the process 2000A in FIG. 20A. For instance, the primary physician Dr. Smith may create the care support group by adding a patient to the care support group (step 2002), adding one or more practitioners to the care support group associated with the patient (step 2004) and adding one or more health records to the care support group associated with the patient (step 2006). The order of steps 2002, 2004, 2006 may vary in various embodiments. The patient-centric EHR system 102 receives the requests from the physician's EHR system 104 to create and add aforementioned aspects to the care support group, processes the request and creates the care support group accordingly which may include creating the data structure 2100. The data structure 2100 is for illustration purposes only and may vary in various implementations of the care support group.

To further illustrate step 2002, the primary physician Dr. Smith may select the interface control to add a patient to the care support group and then may be provided with the information shown in FIG. 19C. FIG. 19C illustrates an example of information shown on the GUI of the primary physician's EHR system 104 for adding a patient to the care support group. As shown, the primary physician Dr. Smith may search for a patient by his/her name, date of birth, health card number and/or any other suitable identifier (e.g., address, phone number, to name a few). Then the primary physician Dr. Smith may add the patient, which in this example is John Doe, to the care support group. The patient-centric EHR system 102 may provide the primary physician's EHR system 104 with a listing of patients. Adding the patient to the care support group may include selecting John Doe's name (or other suitable identifier) from the listing shown on the GUI of potential patients selectable by the physician.

To further illustrate step 2004, the primary physician Dr. Smith may select the interface control to add practitioners to the care support group and then may be provided with the information shown in FIG. 19D. FIG. 19D illustrates an example of information shown on the GUI of the primary physician's EHR system 104 for adding one or more practitioners to the care support group. As shown, the primary physician Dr. Smith may search for a practitioner by his/her name, specialty, location and/or any other suitable identifier (e.g., clinic name, hospital name, to name a few). The patient-centric EHR system 102 may provide the primary physician's EHR system 104 with a listing of practitioners. Then the primary physician Dr. Smith may add a desired practitioner to the care support group. Adding the practitioner to the care support group may include selecting the practitioner's name (or other suitable identifier) from the listing shown on the GUI of potential practitioners selectable by the physician. The primary physician Dr. Smith may add more than one practitioner to the care support group at this step. In this example, the primary physician Dr. Smith adds an oncologist Dr. Johnson, a radiologist Dr. Williams and a nurse practitioner Ms. Jones.

To further illustrate step 2006, the primary physician Dr. Smith may select the interface control to add one or more health records to the care support group and then may be provided with the information shown in FIG. 19E. FIG. 19E illustrates an example of information shown on the GUI of the primary physician's EHR system 104 for adding one or more health records associated with the patient to the care support group. The primary physician Dr. Smith may be provided with a listing of all or a select number of the patient John Smith's health records that are stored on the patient-centric EHR system 102 and then is provided the opportunity to select the health records that the primary physician Dr. Smith believes to be relevant for being added to the care support group for access by the other practitioners associated with the care support group. The selection of the health records for being accessible to the care support group may include the use of a search and/or selection field for selecting specific health records meeting a specific criterion. For example, the primary physician Dr. Smith may be able to select records according to: a date range (e.g., health records before a certain date, after a certain date, between two dates); a location (e.g., a specific clinic, hospital, laboratory, imaging facility, city, province/state, country); a practitioner (e.g., a doctor's or practitioner's name) a condition associated with the record (e.g., disorder, infection, disability, injury, situation and/or any other suitable condition or situation); testing results (e.g., blood tests, urine tests, x-ray images, and/or any other suitable test or image type); and/or any other suitable field for selecting health records.

Once the primary physician Dr. Smith creates the care support group, prior to the other practitioners (which in this example are Dr. Johnson, Dr. Williams and Ms. Jones) are granted access to the care support group the patient may have to grant authorization. At step 2008, the primary physician Dr. Smith requests authorization from the patient to create the care support group. The authorization may be done through the patient-centric EHR system 102 in which case upon creation of the care support group the patient is notified via the patient-centric EHR system 102 of the care support group and is asked to grant access, which may be done according to the process 2000B. In other cases, the authorization may be a verbal authorization by the patient to the physician during the consultation, in which case the primary physician may indicate to the patient-centric EHR system 102 that the patient has provided consent and the other practitioners may now have access to the care support group.

Turning to FIG. 21, the example care support group data structure 2100 stored on the patient-centric EHR system 102 is shown. As shown, the data structure 2100 includes a primary physician identifier 22001 which is used as a pointer to point to a data record 2201 associated with the primary physician Dr. Smith. When the primary physician Dr. Smith creates the care support group (as discussed above) the patient-centric EHR system 102 may create the care support group data structure 2100 and add the primary physician's identifier 22001 upon creation (e.g., creates the pointer). The data record 2201 may include information associated with the primary physician Dr. Smith such that when the primary physician Dr. Smith connects and logs in to the patient-centric EHR system 102 via his EHR system 104, he has access to the care support group associated with the data structure 2100.

The data structure 2100 also includes a patient identifier 11001 which is used as a pointer to point to a data record 2110 associated with the patient John Doe. When the primary physician Dr. Smith created the care support group (as discussed above) and associates the patient to the care support group, the patient-centric EHR system 102 may add the patient identifier 11001 to the data structure 2100 (e.g., creates the pointer). It is appreciated that the patient-centric EHR system 102 may maintain a list of the patients that are registered with the patient-centric EHR system 102 and/or that have health records stored therein. As such, the patient-centric EHR system 102 may have a list of patients where each of the patients is associated with a unique identifier, which may be used in the storage of data and health records associated with each patient. The data record 2110 may include information associated with the patient John Smith such that when the patient John Smith connects and logs in to the patient-centric EHR system 102 via his computing entity 106, he has access to the care support group associated with the data structure 2100. The data record 2110 may include information pertaining to the patient John Smith's health records such as storage of the health records themselves and/or pointers to where the health records associated with the patient may be accessed.

The data structure 2100 also includes other practitioners' identifiers 22012, 22014, 22016 which are used as pointers to point to respective data records 2202 2204 2206 associated with Dr. Johnson, Dr. Williams and Ms. Jones, respectively. When the primary physician Dr. Smith created the care support group (as discussed above) and associates the other practitioners to the care support group, the patient-centric EHR system 102 may add the other practitioners' identifiers 22012, 22014 and 22016 to the data structure 2100 (e.g., creates the pointers). It is appreciated that the patient-centric EHR system 102 may maintain a list of the practitioners that are registered with the patient-centric EHR system 102. As such, the patient-centric EHR system 102 may have a list of all of the practitioners where each of the practitioners is associated with a unique identifier, where the unique identifiers may be used for allowing respective practitioners access to various health records associated with various patients. More specifically, the practitioners' identifiers may be used such that respective practitioners have access to the care support group. The data records 2202, 2204 and 2206 may include information associated with the respective practitioners such that when one of the practitioners connects and logs in (e.g., by providing his/her account identifier and credential) to the patient-centric EHR system 102 via his/her EHR system 104₁, 104₂ or 104₃, he/she has access to the care support group associated with the data structure 2100.

The data structure 2100 also includes health record identifiers 11001-00010, 11001-00011, 11001-00012, 11001-00013, 11001-00014 and 11001-00015, which are used as pointers that point to respective health records 302₁, 302₂, 302₃, 302₄ and 302₅, respectively. When the primary physician Dr. Smith created the care support group (as discussed above) and associates patient's health records to the care support group, the patient-centric EHR system 102 may add the health record identifiers 11001-00010, 11001-00011, 11001-00012,11001-00013, 11001-00014 and 11001-00015 to the data structure 2100. These health records 302₁, 302₂, 302₃, 302₄ and 302₅ may be of the type discussed elsewhere in this document.

Turning now to FIG. 20B, the patient may grant access to the selected practitioners to the care support group according to the process 2000B. At step 2052, the patient-centric EHR system 102 receives the request from the physician's EHR system 104 to create the care support group associated with the patient (e.g., step 2008 of process 2000A). At step 2054, the patient-centric EHR system 102 then notifies the patient John Doe via his computing entity 106 of the request to create the care support group and request authorization from the patient. This step of notification and authorization may be done according to the various methods and process discussed elsewhere in the document, which may include sending a notice which does not provide any confidential information prior to sending the request for authorization. At step 2056, the patient-centric EHR system 102 receives the authorization from the patient, and the patient-centric EHR system 102 authorizes the practitioners associated with the care support group access to patient's health records associated with the care support group. Then at step 2058, in response to the patient's authorization for the creation of the care support group, then each of the practitioners associated with the care support group may have access to the care support group and the associated health records of the patient made available through the care support group.

It is appreciated that the patient may deny access to the creation of the care support group and in such case access would not be granted to the practitioners. In other cases, the patient have the ability in granting the authorization to select which practitioners may be added to the care support group and/or which health records may be added to the care support group. For example, the patient may be provided on the GUI of his computing entity 106 with a list of the other practitioner that the primary physician has added to the care support group and/or a list of the health records that the primary physician has added to the care support group. Then, the patient may be able to select the various practitioners and/or health records that are made available to the care support group.

After the care support group has been created, the patient (e.g., John Doe) may be able to log into the patient-centric EHR system 102 and add additional practitioners and/or health records to the care support group. For example, if the patient John Doe sees another doctor Dr. Anderson, then John Doe may then add Dr. Anderson to his care support group such that Dr. Anderson may be able to log in to the patient-centric EHR system 102 and have access to the care support group. By way of another example, if the patient-centric EHR system 102 receives additional health records associated with the patient, the patient may be notified of the additional health records, which the patient may add to the care support group. In general, the patient may be able to add health records to the care support group by selecting any of the health records stored on the patient-centric EHR system 102 such that the practitioners associated with the care support group have access to the added health records.

In some embodiments, the patient may have the ability to specify which health records to be accessible to which of the practitioners associated with the care support group and may have the ability to place restrictions on the practitioners that have access to the care support group. The practitioners added to the care support group may be added with specific time limitations (e.g., corresponding to a treatment and/or recovery period) and the patient may be able to specify the time periods that one or more of the practitioners has access to the care support group. The patient may also have the ability to be able to log in to the patient-centric EHR system 102 and remove practitioners and/or health records from the care support group.

After step 2058 each of the practitioners may be sent a notice from the patient-centric EHR system 102 that the care support group has been created, that the patient has granted authorization and/or that they now have access to the care support group. In this example, the practitioners Dr. Smith, Dr. Johnson, Dr. Williams and Ms. Jones may then be able to connect to the patient-centric EHR system 102 to have access to the care support group. The practitioners Dr. Smith, Dr. Johnson, Dr. Williams and Ms. Jones may connect via the respective EHR systems 104, 104₁, 104₂ and 104₃. It is appreciated that the practitioners Dr. Smith, Dr. Johnson, Dr. Williams and Ms. Jones may not be tied to a specific system or device and may connect via various EHR systems or devices to the patient-centric EHR system 102 using their account identifiers and credentials.

Each of the practitioners may be able to view the health records associated with the patient that is associated with the care support group. For example, prior to the patient John Doe having a consult with Dr. Johnson, Dr. Johnson may connect to the patient-centric EHR system 102 and view the various records of the care support group associated with the patient John Doe.

Each of the practitioners may be able to add annotations to a specific health record in the group of the health records associated with the care support group. Each of the practitioners may be able to add additional health records to the group of health records associated with the patient that is associated with the care support group. For example, after the patient John Doe has a consult with Dr. Johnson, Dr. Johnson may then add an annotation and/or a new health record to the care support group associated with the patient John Doe, which may detail the Dr. Johnson assessment the patient and/or the patient's current health records.

Continuing with this example, Dr. Johnson may add a treatment regime for the patient John Doe and may add this to a new health record associated the care support group associated with the patient John Doe. Then in administering the treatment regime, the nurse practitioner Ms. Jones may access the care support group associated with the patient John Doe to view the treatment regime which may include a treatment dosage and schedule for the treatment of John Doe's cancer. Ms. Jones may add an annotation and/or new health record to the care support group associated with the patient John Doe after administering the cancer treatment. After adding the annotation and/or new health record related to the patient being administered the treatment, a notice may be sent to Dr. Johnson via the patient-centric EHR system 102 such that Dr. Johnson is notified that the patient is undergoing the prescribed treatment.

It is appreciated that the annotation and/or new health record added to the care support group associated with the patient John Doe may also include the addition of notices and/or action items (which are also discussed elsewhere in this document). For example, Dr. Johnson in adding the treatment regime may add an action item to notify the nurse practitioner that John Doe is going to undergo treatment and that an appointment should be schedule. By way of another example, Dr. Johnson in adding the treatment regime may add an action item such that he will be notified of the patient's treatment. In other words, action items and/or notices may be used by the practitioners to schedule treatments, prescribe prescriptions/treatments, to schedule appointments, setup reminders, inter-practitioner communication, to share laboratory testing and/or imaging results, and/or any other suitable means.

It is also appreciated that the same patient may be part of more than one care support group, as the care support group may be created regarding a specific condition. For example, the patient may have a care support group for the treatment of cancer and another care support group for the treatment of diabetes, where the practitioners in each care support group are different and have different access to the patient's health records. In some cases, the primary physician may be the same in multiple care support groups and some practitioners may be associated with multiple cares support groups associated with the same patient.

Although in this example the plurality of EHR systems 104₁ 104₂ 104₃ associated with other practitioners have EHR functionality, in other embodiments, the systems 104₁ 104₂ 104₃ associated with other practitioners do not have EHR capability are may be any suitable computing system such as a computer, laptop, tablet, mobile phone and/or any other suitable device. In such case, the systems 104₁ 104₂ 104₃ associated with other practitioners may connect to the patient-centric EHR system 102 via a web browser, application software running on the systems 104₁ 104₂ 104₃ and/or any other suitable means.

Although in this example, the primary physician Dr. Smith creates the care support group, in other embodiments, the patient or other practitioners may create the care support group in a similar fashion to that described herein.

Although in the this example, the primary physician Dr. Smith added specific practitioners, in other examples the primary physician Dr. Smith may have added a facility to the care support group. For example the facility added to the care support group may be a laboratory or testing facility, which can then view the test prescribed by any of the practitioners in the care support group and then add the test results to the care support group. In other words, access to the care support group may be assigned to specific entities such as hospitals, clinics, laboratories and/or any other suitable entity.

In other examples, the primary physician Dr. Smith may also add the computer based decision support agent (discussed elsewhere in this document) to assess the patient's health record such that the practitioners associated with the care support group may view the results from the decision support agent, which may then be used for the treatment of the patient and/or discussed among the practitioners.

It is further appreciated that the care support group may also be used for clinical research.

Anonymized Health Record Access Mechanism

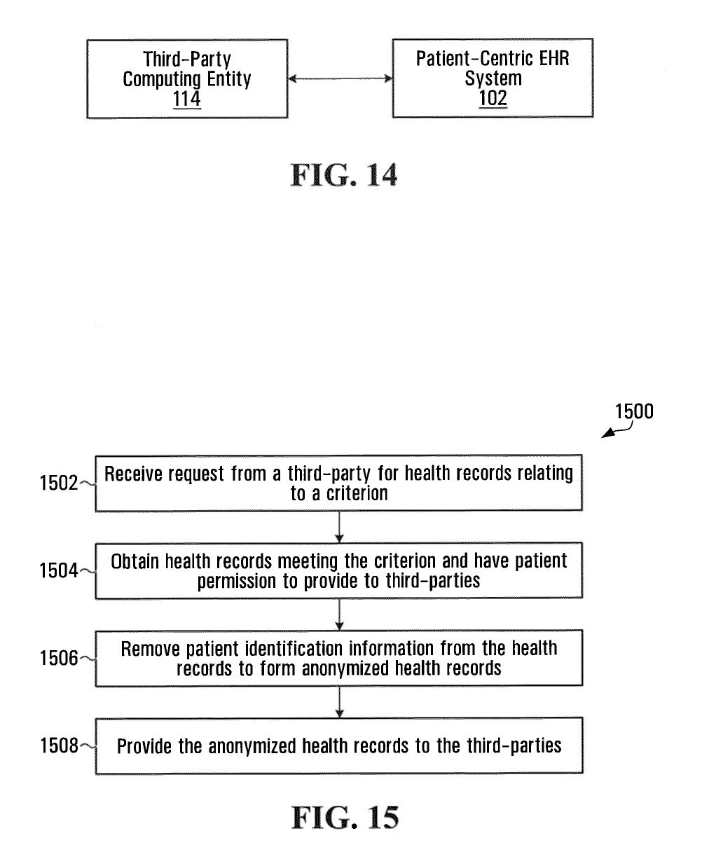

FIG. 14 illustrates a block diagram of an example of the patient-centric EHR system 102 connected to an agent or third-party computing entity 114, such that the patient-centric EHR system 102 may provide patient anonymized health records to a third-party computing entity 114. In other cases, the health records provided to the third-party computing entity 114 are not anonymized and may be similar to the various other embodiments discussed in this document. The connection between the patient-centric EHR system 102 and the third party computing entity 114 may include one or more connections over one or more data networks. The connection between the patient-centric EHR system 102 and the third party computing entity 114 may allow for the communication (e.g., transmission and/or reception) of various information and/or data between the various systems and/or devices. The various information and/or data exchanged may be a “notice”, “notices” a “health record” and/or “health records” of the type discussed elsewhere in this document.

FIG. 15 illustrates a flowchart of a process 1500 for providing patient anonymized health records to the third-party computing entity 114. At step 1502, the patient-centric EHR system 102 receives a request from a third-party for health records relating to a criterion or criteria. In this example, the third party is the third-party computing entity 114. Then, at step 1504, the patient-centric EHR system 102 obtains health records meeting the criterion or criteria, by processes the criterion or criteria against the database 206 of health records to obtain health records meeting the criterion or criteria and where each obtained record has permission from the respective patient associated with the record for the record to be provided to third-parties. For instance, when patients register with the patient-centric EHR system 102 they may be asked if they are willing to provide anonymized data from their health record to third-parties. In other words, a patient may be able to deny access to third-parties to his/her health record. For example, as is shown in FIG. 16E, there may be a settings page associated with the patient's account with the patient-centric EHR system 102, such that the patient can view this page once he/she is logged into the patient-centric EHR system 102. Then at step 1506, the patient identification information is removed from the health records to form a cohort of anonymized health records. At step 1508, the cohort of anonymized health records is provided to the third-party computing entity 114.

In some embodiments, the third-party computing entity 114 is associated with an agent that is associated with the patient-centric EHR system 106. For example, the operator or the patient-centric EHR system 106 could have agreements with agents to provide agents with access to various aspects of the data stored in the patient-centric EHR system 106. For example, in one case, an agent could be registered to obtain a cohort of patient health records meeting a certain criteria. The patient-centric EHR system 106 upon a query could provide anonymized data to the third-party computing entity 114, such that the agent could conduct research with the cohort of patient health records. The patient can allow/deny being discovered as part of a query for cohorts. In order for the patient-centric EHR system 106 to allow for the privacy of patient health information, the patient may be able to configure via the patient's computing entity 106 whether he/she is allowed to be discovered by queries from third-parties. If the patient desires for his/her medical information to not be provided to third-parties, even if the patient matches the criteria of the query, his/her anonymized data is not returned to the third-party agent.

In some embodiments, if patient agrees to provide anonymized data to third-parties, each time his/her name is picked in a query (e.g., on a per a study basis), he/she may have the option to opt-out of the cohort. For example, each time his/her name is picked in a query, he/she may receive a notice (such as the type discussed elsewhere in this document) and accept or deny his/her health records to be included in the cohort.

In some embodiments, the agent is able to select a specific anonymized patient from the cohort of patient health records to conduct further health analysis (diagnostics) or establish patient membership in a specific research topic. For example, if a specific anonymized patient, he/she may receive a notice (such as the type discussed elsewhere in this document) and accept or deny his/her health record to be included in the further health analysis (diagnostics) or establish patient membership in a specific research topic

In some embodiments, the agent could be a ‘blood pressure’ monitor software-as-a-service component, that could alert the patient (and physician) of an abnormal situation over time. The patient again may subscribe, permit notifications, and the physician can do the same for certain ‘thresholds’ defined by the agent. The ‘blood pressure monitor’ may not need to be co-resident with the patient-centric EHR system, but may makes use of notifications and notification-lists to habilitate and regulate the flow of information device-agent-medical record-physician.

In some embodiments, the patient-centric EHR system 102 could have agreement with agents for ‘travel related risks’. In this case, the patient subscribes for notifications from the agent and authorizes the agent to use his/her information to monitor potential health hazards as he moves around the globe. In this case, the data is not anonymized, since it is used directly for a specific patient.

The patient-centric EHR system 102 may include in the account of the patient a record of the various studies and/or third-parties that he/she participated in, which may be used for compensating the patient for participating. The patient may be compensated monetarily or may be compensated by receiving various information and/or data from the study that may be useful to the patient. For instance, the patient-centric EHR system 102 may notify the patient via the patient's computing entity 106 with a notice that a third-party is requesting to use his/her data and provide him/her with the available compensation, if the patient accepts to provide his/her health record. If the patient acknowledges by selecting the allow option in the notice, then the patient's account may be compensated.

The term “agent” may be used to refer to a third-party system that may be able to receive one or more health records associated with a patient, where the health records may or may not be anonymized. As such, the patient may provide one or more health records to an agent where the agent could provide monitoring, processing or computational service of heath records. It is appreciated that the agent may include the computer based decision support agent discussed elsewhere in this document.

In another embodiment, the patient-centric EHR system 102 may be used to provide a decision aid for physicians. For example, the physician at the physician's EHR system 104 may be able to configure various parameters of a health record associated with a patient on the patient-centric EHR system 102. Such parameters including which record and which information may be made available to third-parties. As such the physician may then provide a health record associated with a patient or various parts of the patient's health record that the physician would like to provide to a third-party such as an agent (e.g., the agent at the third-party system 111 or third-party computing entity 114). More specifically, the physician may provide one or more workflows to be done by the agent on the physician's behalf. Furthermore, the physician may be able to configure the “anomaly” detection thresholds of various observations to suit his/her practice. Also, an agent may be able to examine a patient's entire records collection, and evaluate observations against each other and against the patient's health condition, to try to determine whether some adverse condition is possible given the patient's observation results and other information available therein. In some cases, the agent is another physician that the primary physician would like to have his/her patient's health record reviewed by.

Although the term electronic health record (EHR) system is used in this document, this term may also be referred to as an electronic health or medical record system or any other suitable termed system, computing entity or computing platform.

Any feature of any embodiment discussed herein may be combined with any feature of any other embodiment discussed herein in some examples of implementation.

Any of the steps of the processes discussed herein in may be done in different orders, the steps of process discussed herein may be combined and some steps of the processes discussed herein may be omitted in some examples of implementation.

The use of the term “patient” is used herein to refer to a person or an individual and is not intended to be limiting. For example, term “patient” could be used to include a legal guardian acting on behalf of the patient.

In the embodiments discussed herein the term “physician” is used, in other examples of implementation other types of medical professions, such as dentists, optometrists, pharmacists, nurses, nurse practitioners, physician assistants and/or any other suitable medical professional may be used synonymously with the term “physician”. The term “physician” may include any medical or health related “practitioner”, where the practitioner include may include any person that has access to read and/or write records to a patient's record.

Although reference is made in the examples above where interaction takes place with a government-managed EHR system and/or network, in other examples, other EHR systems and/or networks associated with other organizations (e.g., commercial organizations, government health care systems, HMOs, etc.) may synonymously be used.

It is also appreciated that the term database when referenced in this document could be a single structured table that includes information or it could reference to a collection of databases that could have multiple records or tables that can work jointly or independently of each other. In other words, the reference to database in this document may be to indicate the function of storage or reception of information such as patient records, summary medical records, prescription information, drug information, patient information, insurance information, data records, health records, medical records, etc. in one or more database, one or more tables and/or one or more records, where the databases, tables, and/or records are stored in one or more computer readable memories.

It is further appreciated that the term “EHR system” may be interpreted to include any computer based system that runs or accesses application software that provides the functionality of electronic record systems described herein. For instance, the application software may be running on the EHR system itself or may be running on a separate computer system or server arrangement that the EHR system accesses (e.g., over a data network).

Certain additional elements that may be needed for operation of some embodiments have not been described or illustrated as they are assumed to be within the purview of those of ordinary skill in the art. Moreover, certain embodiments may be free of, may lack and/or may function without any element that is not specifically disclosed herein.

Although various embodiments and examples have been presented, this was for the purpose of describing, but not limiting, the invention. Various modifications and enhancements will become apparent to those of ordinary skill in the art and are within the scope of the invention, which is defined by the appended claims.

1. A method for populating an electronic health record system with health records associated with respective patients, the method comprising:
a. querying a centralized health record network storing health records about multiple patients, the querying being performed in the context of a physician dispensing medical services to a first patient of the multiple patients, the querying including accessing the health record of the first patient;
b. copying some or all of the information in the health record of the first patient to the electronic health record system to create health record for the first patient in the electronic health record system;
c. repeating steps (a) and (b) for a second patient of the multiple patients;
d. wherein the physician dispenses medical services to only a subset of patients of the multiple patients.

2. The method of claim 1, including providing an account to the first patient such that the first patient can access the health record associated therewith on the electronic health record system.

3. A method for providing health records associated with a patient to an electronic health record system, the method comprising:
a. receiving an indication for a request to obtain the health records associated with the patient;
b. requesting the health records associated with the patient from an electronic health record network comprising a plurality of nodes, the health records associated with the patient being distributed amongst the plurality of nodes of the health record network;
c. obtaining the health records associated with the patient, in response to requesting the health records associated with the patient from the electronic health record network; and
d. transmitting the health records associated with the patient to an electronic health record system for centralized storage of the health record.

4. The method of claim 3, wherein the indication for the request to obtain the health records associated with the patient from the electronic health record network is received from a patient-centric electronic health record system or is received by the patient-centric electronic health record system.

5. The method of claim 3, wherein the electronic health record system for centralized storage of the health records is a patient-centric electronic health record system.

6. The method of claim 3, wherein the plurality of nodes of the electronic health record network is a plurality of electronic health record systems.

7. The method of claim 6, wherein said requesting the health records associated with the patient from the electronic health record network comprises requesting from a server associated with the plurality of electronic health record systems to obtain the health records associated with the patient from the plurality of electronic health record systems.

8. The method of claim 5, wherein said requesting the health records associated with the patient from the electronic health record network comprises requesting from each of the plurality of electronic health record systems to obtain the health records associated with the patient from the plurality of electronic health record systems.

9. A method for providing at least one computing device associated with a patient a notice that medical results associated with the patient have been received at an office associated with a physician, the method comprising:
a. receiving from an electronic record system associated with the physician an indication that medical results associated with the patient have been received at the office associated with the physician;
b. transmitting to a computing entity associated with the patient an first notice that medical information is available;
c. receiving a request for the medical information from the computing entity associated with the patient, the request including authentication information; and
d. if the authentication information is associated with the patient, transmitting to the computing entity associated with the patient a second notice, the second notice indicating that medical results associated with the patient have been received at the office associated with the physician.

10. A method for providing medical results associated with a patient to a computing device associated with the patient, the method comprising:
a. receiving medical results associated with the patient from an electronic record system associated with a physician;
b. transmitting to a computing entity associated with the patient an first notice that medical information is available;
c. receiving a request for the medical information from the computing entity associated with the patient, the request including authentication information; and
d. if the authentication information is associated with the patient, transmitting to the computing entity associated with the patient a second notice, the second notice comprising the medical results associated with the patient.

11. The method of claim 10 wherein the medical results comprise medical results from a third-party medical facility.

12. The method of claim 10 wherein the medical results comprise an annotation from the physician.

13. The method of claim 10 wherein the medical results comprise an action item.

14. The method of claim 13, wherein the action item is to schedule an appointment with the physician.

15. The method of claim 13, wherein the action item is a prescription.

16. A method for making a health related determination, the method comprising:
a. monitoring data obtained from one or more sensors measuring a physical condition of a patient, the monitoring of the data from the one or more sensors being done over a plurality of time points;
b. storing the data from the one or more sensors in association with medical records associated with the patient
c. processing the data from the one or more sensors against the medical records associated with the patient to make a health related determination, the medical records being obtained from a plurality of electronic health record systems.

17. The method of claim 16, wherein the one or more sensors measure blood glucose and the health related determination is that the blood glucose level of the patient is high.

18. The method of claim 16, wherein the one or more sensors measure heart rate and the health related determination is that the heart rate of the patient is high.

19. The method of claim 16, wherein the one or more sensors measure blood pressure and the health related determination is that the blood pressure of the patient is high.

20. The method of claim 16 further comprising transmitting a notification of the health related determination to a computing entity associated with the patient.

21. The method of claim 16 further comprising transmitting a notification of the health related determination to an electronic health record system associated with a physician.

22. A method for providing a third-party with health records associated with a patient, the method comprising:
a. receiving a request to provide the third-party with the health records associated with the patient;
b. authenticating the request with a computing device associated with the patient;
c. providing a third-party computing device with access to the health records associated with the patient.

23. The method of claim 22 wherein the request is received from the computing device associated with the patient.

24. The method of claim 22 wherein the request is received from the computing device associated with the third-party.

25. The method of claim 22 wherein the access to health records is provided for a limited time period.

26. The method of claim 21, wherein the method further comprises receiving from the computing device associated with the third-party an annotation associated with one or more of the health records associated with the patient.

27. A method of providing health records to an agent, the method comprising:
a. receiving a request for a plurality of health records meeting a specific criteria from a third-party computing entity associated with the agent;
b. obtaining a plurality of health records meeting the specific criteria; and
c. providing to the third-party computing entity the plurality of health records.

28. The method of claim 27 further includes removing patient identifying characterizes from the plurality of health records meeting the specific criteria to form a plurality of anonymized health records prior to providing the third-party computing entity the plurality of health records, wherein the plurality of health records provided to the third-party are anonymized.
